(19) United States
(12) Patent Application Publication
(10) Pub. No.: US 2010/0168616 A1
(43) Pub. Date: Jul. 1, 2010

(54) LANCET DEVICE UTILIZING A REVOLVER-TYPE CARTRIDGE, REVOLVER-TYPE CARTRIDGE, AND METHOD OF MAKING AND/OR USING THE CARTRIDGE AND THE LANCET DEVICE

(75) Inventors: Steven Schraga, Surfside, FL (US); Paul R. Fuller, Danvers, MA (US); Brian Schwartz, Lake in the Hills, IL (US); David A. Carhart, Cary, IL (US); Steve Fragassi, Kenosha, WI (US)

Correspondence Address: GREENBLUM & BERNSTEIN, P.L.C., 1950 ROLAND CLARKE PLACE, RESTON, VA, 20191, US

(73) Assignee: STAT MEDICAL DEVICES, INC., North Miami, FL (US)

(21) Appl. No.: 12/515,724

(22) PCT Filed: Nov. 21, 2007
(86) PCT No.: PCT/US07/85422

§ 371 Date: Mar. 17, 2010

Related U.S. Application Data
(60) Provisional application No. 60/860,228, filed on Nov. 21, 2006.

(51) Int. Cl. A61B 5/00; A61B 17/32

(52) U.S. Cl. 600/583; 606/182

(57) ABSTRACT

A cartridge for a device includes a generally cylindrical cartridge body. A plurality of generally axially movably mounted lancets or lancet needles are included. A plurality of biasing members are utilized with each being adapted to apply a biasing force to one of the lancets or lancet needles. A lancet device includes a housing comprising a trigger and a generally cylindrically-shaped cartridge comprising a plurality of lancet needles and a plurality of springs and being adapted to rotate between different positions.

### CROSS-REFERENCE TO RELATED APPLICATIONS

The instant application is an International Application based on U.S. provisional application No. 60/860,228, filed Nov. 21, 2006, the disclosure of which is hereby expressly incorporated by reference hereto in its entirety.

### BACKGROUND OF THE INVENTION

1. Field of the Invention

The invention relates to a cartridge which includes a plurality of movably mounted lancets or lancet needles. The invention also relates to a disposable revolver-type cartridge for a lancet device. The invention further relates to a method of using a lancet device with a removable/replaceable cartridge. In particular, the invention relates to a cartridge having lancet needles and which may be disposable, i.e., which cartridge can be used once and discarded, and/or which utilizes an arrangement which protects a user from contacting his or her skin with the lancet needles after the cartridge has been used. The invention also contemplates using a revolver-type cartridge on various types of lancet devices as well as various types of testing devices such as, e.g., glucose testing meters.

2. Discussion of Background Information

Lancet devices are commonly used to prick the skin of the user so that one or more drops of blood may be extracted for testing. Some users, such as diabetics, for example, may have to test their blood sugar levels several times a day. This may be accomplished by the user using a simple needle. However, this procedure is often problematic for the user since the needle may be difficult to handle. Additionally, many users simply cannot perform the procedure owing to either a fear of needles or because they lack a steady hand. As a result, lancet devices have been developed which allow the user to more easily and reliably perform this procedure.

Known single-use/disposable lancet devices are not sufficiently and/or properly design to ensure that they cannot be reused. Moreover, such devices generally do not protect a user from coming into contact with body fluids such as blood which may be on the device after the device has been used.

An improved device would allow the user to use the lancet needle only a single time and more reliably and safely prevent reuse of the lancet needle. The device should also ensure that a contaminated surface of the lancet needle(s) cannot come into contact with a user after the device is used. Finally, an improved device would utilize a cartridge which is safe to dispose of, is simple in design, and is inexpensive to produce.

### SUMMARY OF THE INVENTION

According to one illustrative aspect of the invention there is provided a disposable cartridge for devices such as, e.g., a lancet device, a testing device, and a glucose meter. The cartridge may be a drum-shaped revolver type magazine cartridge and can includes two, three, four, five lancets, etc, i.e., any number of lancets between two and 12 or more.

According to another illustrative aspect of the invention there is provided a cartridge for a lancet device, wherein the cartridge comprises a plurality of lancet needles and a mechanism allowing the cartridge to be mounted to the lancet device.

The lancets may be individually moved to an extended position by a pushing device which merely contacts and pushes the lancet without becoming connected to the lancet in any way, temporarily or otherwise. The needles of the lancets can be axially oriented in a manner which is offset from a main axis of the lancet. The mechanism may allow the cartridge to be non-axially movably mounted to the lancet device and may comprise one or more projecting members and/or biasing devices. The mechanism may allow the cartridge to be non-axially and rotatably mounted to the lancet device and may comprise one or more projecting members and/or biasing devices. The mechanism may allow the cartridge to be removably mounted to the lancet device and may comprise one or more projecting members and/or biasing devices. The mechanism may allow the cartridge to be non-axially movably mounted, rotatably mounted, and removably mounted to the lancet device and may comprise one or more projecting members and/or biasing devices. Each of the plurality of lancet needles may be generally axially oriented, i.e., oriented generally parallel to a central and/or rotatable axis of the cartridge. The cartridge may further comprise a plurality of biasing members that are generally axially oriented. Each of the plurality of lancet needles may be movably mounted to a revolver-shaped body, i.e., a revolving cylinder having axially oriented openings. Each of the plurality of lancets may be biased by biasing members towards a retracted position. The cartridge may comprise a generally cylindrical shape whose axial length is greater than its overall diameter. The cartridge may comprise a generally cylindrical shape having an outer diameter of no greater than about 1 inches. The cartridge may comprise a generally cylindrical shape having an axial length of no greater than about 1.5 inches.

The cartridge may further comprise a plurality of springs, wherein each spring is mounted to one of the plurality of lancet. Each of the plurality of lancets may comprise a head portion and a needle portion. The cartridge may further comprise a revolver-shaped cartridge, wherein each needle portion is arranged within an opening. The cartridge may further comprise an alignment mechanism allowing the cartridge to be mounted to the lancet device in only a single position. The cartridge may further comprise one of a notch, a groove, and a projection which ensures that the cartridge is mounted to the lancet device in a predetermined position. The cartridge may further comprise a locking mechanism preventing rotation of the cartridge once the cartridge has rotated in the lancet device to a locking position.

The invention also provides for a cartridge for a device, wherein the cartridge comprises a generally cylindrical cartridge body, a plurality of generally axially movably mounted lancets or lancet needles, and a plurality of biasing members, each adapted to apply a biasing force to one of the lancets or lancet needles.

The cartridge may further comprise a mechanism allowing the cartridge to be mounted to the device. The device may comprise at least one of a lancet device, a testing device, and a glucose meter. The cartridge may be adapted to rotate about a central axis. The cartridge may be adapted to rotate in only one direction about a central axis. The biasing members may comprise springs, each spring having one end adapted to contact one of the plurality of lancets or lancet needles. The cartridge may comprise an axial length which is greater than an outer diameter. The cartridge may comprise an outer diameter of no greater than about 1 inch. The cartridge may comprise an axial length of no greater than about 1.5 inches. Each lancet or lancet needle may comprise an opening or recess which receives therein a portion of one of the biasing members. Each of the plurality of lancets or lancet needles may comprise a synthetic resin head portion and a metal needle portion. The cartridge may comprise a plurality of equally angularly spaced openings each sized to receive therein one of the lancets or lancet needles. The cartridge may further comprise a centrally arranged opening sized to receive therein a device for causing rotation of the cartridge. The cartridge may further comprise a centrally arranged opening comprising at least one of an axial groove and an axial projection adapted to engage with a device for causing rotation of the cartridge. The cartridge may further comprise a centrally arranged opening comprising axial grooves and projections adapted to engage with a spline of a device for causing rotation of the cartridge. The cartridge may further comprise a plurality of openings arranged on back end of the cartridge, each opening being sized to receive therein a device for causing one of the lancets or lancet needles to move to an extended or puncturing position. The device for causing one of the lancets or lancet needles to move to an extended or puncturing position may contact a rear-most end of the one of the lancets or lancet needles. The one of the lancets or lancet needles may automatically move back to a retracted position when the device for causing one of the lancets or lancet needles to move to an extended or puncturing position is moved to a retracted position. The cartridge may further comprise an alignment mechanism allowing the cartridge to be mounted to the device in only a single position. The cartridge may further comprise one of a notch, a groove, and a projection which ensures that the cartridge is mounted to the device in a predetermined position. The cartridge may further comprise a locking mechanism preventing rotation of the cartridge once the cartridge has rotated in the device to a locking position. The cartridge may further comprise a projection which prevents rotation of the cartridge after the cartridge has rotated in the device to a final position.

The invention also provides for a method of puncturing a surface of skin using a device comprising the cartridge of the type(s) described above, wherein the method comprises arranging the device adjacent against a user's skin, triggering the device so that one of the plurality of lancets or lancet needles is caused to penetrate the user's skin, and rotating the cartridge to another position.

The invention also provides for a cartridge for a device, wherein the cartridge comprises a generally cylindrical-shaped body, a plurality of axially oriented lancet needles, a plurality of springs, and each spring being structured and arranged to move one of the plurality of lancet needles towards a retracted position, wherein the cartridge is structured and arranged to be at least one of rotatably mounted, removably mounted, and non-axially movably mounted to the device.

The generally cylindrical-shaped body may comprise a center opening and a plurality of axially oriented and equally angularly spaced openings, wherein the plurality of axially oriented lancet needles are movable along a generally linear path without substantially rotating, and wherein the center opening is adapted to receive a device for causing rotation of the cartridge.

The invention also provides for a method of puncturing a surface of skin using a device comprising the cartridge of the types described above, wherein the method comprises arranging the device adjacent against a user's skin, triggering the device so that one of the plurality of lancet needles is caused to penetrate the user's skin, and rotating the cartridge to another position.

The invention also provides for a lancet device comprising a housing comprising a trigger and a generally cylindrically-shaped cartridge comprising a plurality of lancet needles and a plurality of springs and being adapted to rotate between different positions.

The cartridge may comprise a center opening which is sized to receive therein a portion of a device for causing rotation of the cartridge. The plurality of lancet needles may be independently movably mounted. The plurality of lancet needles may be generally axially oriented. The cartridge may be removably mounted to the housing. The cartridge may be insertable into the housing in only one rotatable position and removable from the housing in only one rotatable position. The cartridge may be removable from the housing in only a final rotatable position after all of the lancet needles have been utilized.

The invention also provides for a device comprising a housing, a cartridge comprising a plurality of lancet needles, the cartridge being rotatably mounted to a front end of the housing, a mechanism which prevents axial movement of the cartridge after the cartridge is mounted to the front end of the housing, and a device rotatably mounted to the housing which allows a user to rotate the cartridge between a plurality of different positions, wherein the device has one end which extends outside of a rear end of the housing and is adapted to be gripped by the user to cause rotation thereof.

The housing may comprise a generally cylindrically shaped two-piece housing. The device may further comprise a mechanism which retains the cartridge in at least one rotational position. The device which allows a user to rotate the cartridge between a plurality of different positions may comprise a spline and is axially movable at least between an initial position and a position which places the device in an armed or trigger-set position. The device may further comprise a mechanism which retains the cartridge in at least one rotational position, wherein the mechanism which retains the cartridge in at least one rotational position comprises a deflecting member arranged on the device rotatably mounted to the housing. The device which allows a user to rotate the cartridge between a plurality of positions may be biased towards an initial position. The device may further comprise an alignment mechanism allowing the cartridge to be initially mounted within the front end of the housing in only a single position. The device may further comprise a deflectable projection which ensures that the cartridge is mounted to the housing in a predetermined position. The device may further comprise guiding grooves arranged in the front of the housing for allowing insertion and rotation of the cartridge. The cartridge and the housing may comprise an arrangement which prevents rotation of the cartridge after the cartridge has rotated in the housing to a predetermined position. The device may further comprise a depth adjustment cap arranged at the front end of the housing.

The invention also provides for a method of puncturing a surface of skin using the device of the types described above, wherein the method comprises arranging the device adjacent against a user's skin, triggering the device so that one of the plurality of lancet needles is caused to penetrate the user's skin, and rotating the cartridge to another position.

According to another illustrative aspect of the invention there is provided a cartridge for a device, wherein the cartridge comprises a revolver-shaped cartridge body comprising a plurality of axially oriented lancet and/or lancet needles, wherein the cartridge is mountable to the device. Wherein each of the lancets or lancet needles move along an axis which is generally parallel to a central axis of the cartridge and spaced from the central axis.

According to another illustrative aspect of the invention there is provided a lancet device which can be armed and fired with one hand, whereby the user arms the device with his or her thumb and fires the device with his or her index finger. The device can utilize a cartridge which contains plural lancets, e.g., 5 lancets, and which can be slid into a front end of the lancet device with one of the user's hands while the other hand grips the lancet device housing and then armed and fired with the hand gripping the lancet device.

The invention also provides for a method of assembling a device for puncturing a skin surface, wherein the method comprises installing a cartridge comprising a plurality of lancets and lancet springs at least partially within a device housing, whereby the cartridge, once installed, is capable of being rotated between at least two different positions.

The invention also provides for a method of using a device for causing a puncture in a skin surface, wherein the method comprises installing a cartridge comprising a plurality of lancets and lancet springs at least partially within a device housing, triggering the device so that one of the plurality of lancet needles is caused to penetrate the user's skin, and rotating the cartridge to another position.

Other exemplary embodiments and advantages of the present invention may be ascertained by reviewing the present disclosure and the accompanying drawing.

### BRIEF DESCRIPTION OF THE DRAWINGS

The present invention is further described in the detailed description which follows, in reference to the noted plurality of drawings by way of non-limiting examples of exemplary embodiments of the present invention, in which like reference numerals represent similar parts throughout the several views of the drawings, and wherein:

FIG. 1 shows a side cross-section view of a non-limiting embodiment of a lancet device utilizing a revolver-type cartridge. The device is shown in a static or initial state which occurs during shipping, storing, and after one of the lancets is fired;

FIG. 2 shows another side cross-section view of FIG. 1 with the device shown in a loading state which occurs when a user presses down in the rounded rear end of the arming member;

FIG. 3 shows another side cross-section view of FIG. 1 with the device shown in an armed state which occurs when a user allows the rounded rear end of the arming member to move back to the position shown in FIG. 1;

FIG. 4 shows another side cross-section view of FIG. 1 with the device shown in a triggered state which occurs when a user presses down on the trigger button. One of the lancet needles is shown in an extended or puncturing position;

FIG. 5 shows a side perspective view of the lancet device shown in FIG. 1 with the lower half of the housing removed and with various internal features removed to more clearly illustrate how the arming member is coupled to the cartridge via a spline arranged on the front end of the arming member so that rotation of the arming member causes rotation of the cartridge;

FIG. 6 shows an enlarged front portion of FIG. 5;

FIG. 7 shows a cartridge of FIG. 1 removed from the housing. For purposes of illustration, one of the lancets is shown in the fully extended position (as indicated by one of the lancet needles projecting out of the front end of the cartridge). The lancet needles are normally prevented from assuming the extended position when the cartridge is removed from the housing;

FIG. 8 shows a front portion of the upper housing shown in FIGS. 5 and 6;

FIGS. 9 and 10 respectively show a front section view and a front side perspective view of a rear portion of lancet device shown in FIG. 1, and illustrate how the arming member can be retained in five rotational indexing positions. In FIG. 9, the deflectable projection of the arming member is positioned in the recess of the upper housing located at about the 11:00 O'clock position. The five recesses, which can temporarily rotationally retain the deflectable projection, are equally angularly spaced by about 72 degrees;

FIGS. 11-13 show various views of the trigger area of the lancet device shown in FIG. 1 with the trigger being shown transparent for purposes of illustration. FIG. 11 shows the trigger position when the lancet device is in the static position shown in FIG. 1. FIG. 12 shows the trigger position when the lancet device is in the armed position shown in FIG. 3. FIG. 14 shows the trigger position when the lancet device is in the loading position shown in FIG. 2;

FIGS. 14-16 show various views of the arming member used in the lancet device of FIG. 1. FIG. 14 shows a rear side perspective view of the arming member. FIG. 15 shows a front side perspective view of the arming member. FIG. 16 shows a side cross-section view of the arming member;

FIGS. 17 and 18 show various views of a plunger assembly used in the lancet device of FIG. 1. FIG. 17 shows a rear side perspective view of the plunger assembly. FIG. 18 shows a side cross-section view of FIG. 17;

FIG. 19 shows a rear side perspective view of a forward plunger member used in the plunger assembly shown in FIGS. 17 and 18;

FIG. 20 shows a front side perspective view of the forward plunger member shown in FIG. 19;

FIG. 21 shows a rear side perspective view of a read plunger member used in the plunger assembly shown in FIGS. 17 and 18;

FIG. 22 shows another rear side perspective view of the rear plunger member shown in FIG. 21;

FIG. 23 shows a rear side perspective view of a trigger slide member used in the lancet device shown in FIG. 1;

FIG. 24 shows another a front side perspective view of the trigger slide member shown in FIG. 23;

FIG. 25 shows a front side perspective view of a return spring used in the lancet device shown in FIG. 1;

FIG. 26 shows a front side perspective view of a drive spring used in the lancet device shown in FIG. 1;

FIG. 27 shows a front side perspective view of an arming spring used in the lancet device shown in FIG. 1;

FIG. 28 shows a front side perspective cross-section view of the cartridge assembly used in the lancet device shown in FIG. 1 and shows one of the lancets and the lancet springs in cross-section located in an initial or retracted positioned;

FIG. 29 shows a front side perspective view of the cartridge assembly used in the lancet device shown in FIG. 1;

FIGS. 30-32 show various views of the cartridge body used in the cartridge assembly shown in FIGS. 28 and 29. FIG. 30 shows a rear side perspective view of the cartridge body. FIG. 31 shows a front side perspective cross-section view of the cartridge body. FIG. 32 shows a front side perspective view of the cartridge body;

FIG. 33 shows a front side perspective view of one of the five lancets used in the cartridge assembly shown in FIGS. 28 and 29;

FIG. 34 shows a rear side perspective view of the lancet shown in FIG. 33;

FIG. 35 shows a rear side perspective view of a cartridge retainer member used in the cartridge assembly shown in FIGS. 28 and 29;

FIG. 36 shows a front side perspective view of the cartridge retainer member shown in FIG. 35;

FIG. 37 shows a rear side perspective view of an upper housing portion of the lancet device shown in FIG. 1;

FIG. 38 shows a rear inside perspective view of the upper housing portion shown in FIG. 37;

FIG. 39 shows a rear side perspective view of a lower housing portion of the lancet device shown in FIG. 1;

FIG. 40 shows a rear inside perspective view of the lower housing portion shown in FIG. 39;

FIG. 41 shows a front side perspective view of a trigger member used on of the lancet device shown in FIG. 1;

FIG. 42 shows a front inside perspective view of the trigger member shown in FIG. 41;

FIG. 43 shows a front side perspective view of a depth adjustment inner sleeve used on of the lancet device shown in FIG. 1;

FIG. 44 shows a rear side perspective view of the depth adjustment inner sleeve shown in FIG. 43;

FIG. 45 shows a front side perspective view of a depth adjustment outer sleeve used on of the lancet device shown in FIG. 1;

FIG. 46 shows a rear side perspective view of the depth adjustment outer sleeve shown in FIG. 45;

FIG. 47 shows a front side perspective view of a front cap used on of the lancet device shown in FIG. 1;

FIG. 48 shows a rear side perspective view of the front cap shown in FIG. 47;

FIG. 49 shows a front side perspective view of a front portion of the lancet device shown in FIG. 1 and illustrates how the members (shown transparent for purposes of illustration) shown in FIGS. 43-48 are mounted to the front end of the housing of the lancet device;

FIG. 50 shows a rear side perspective view of the lancet device shown in FIG. 1 and illustrates how the lancet device can be armed and triggered using only one hand of the user. As this figure demonstrates, the user can place his or her thumb on the rear button portion of the arming member and depress it in the direction of arrow. Then, the user can depress the trigger with the user's index finger to cause the lancet device to automatically move one of the lancets to the extended or puncturing position;

FIGS. 51 and 52 show two side views of the lancet device shown in FIG. 50 with FIG. 52 showing the lancet device of FIG. 51 rotated upwards by about 90 degrees;

FIGS. 53 and 54 show front portions of the lancet device shown in FIG. 50 and illustrate how the user can adjust a depth of penetration of the lancet needle. FIG. 53 shows how the outer sleeve of FIGS. 45 and 46 can be rotated relative to the housing so as to cause axial movement of the inner sleeve of FIGS. 43 and 44 which, in turn, moves the front cap of FIGS. 47 and 48 axially away from a front end of the cartridge assembly. The outer sleeve is shown transparent for purposes of illustration; and

FIG. 55 shows a side cross-section view of the cartridge assembly used in the lancet device shown in FIG. 1 and illustrate how an axis of the main body portion of each lancet is axially offset from a center axis of the lancet needle and that both of these axes are axially offset from a center axis of the cartridge body.

### DETAILED DESCRIPTION OF THE PRESENT INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

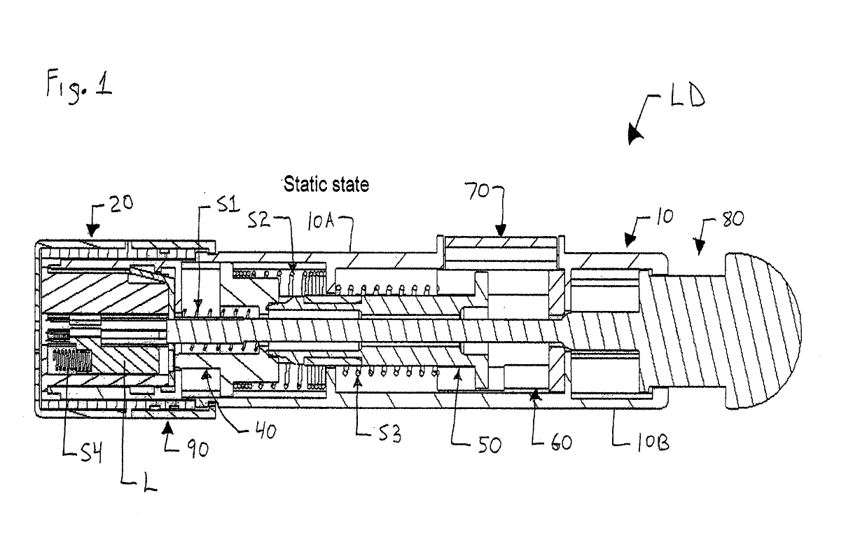

FIGS. 1-55 show one non-limiting embodiment of a lancet device LD. The lancet device LD includes the following main components: a body 10, a front assembly 90, a front cap 20, a barrel or revolver-type cartridge 30, a forward or front plunger member 40, a rear plunger member 50, a trigger slide member 60, a trigger 70, an actuating member cap 80, and three springs S1, S2 and S3.

As can be seen in FIGS. 1-4, the lancet device LD functions as follows: In the position shown in FIG. 1, the lancet device LD is shown in the static or initial position. This is the preferred position that the device would assume during shipping, storage, and after the device is triggered or fired. In this position, the return spring S1 is in a relaxed or non-compressed position. The drive spring S2 and arming spring S3 are preferably in a slightly compressed position with the arming spring S3 applying a biasing force against a front surface of the trigger slide member 60 which in, in turn, places the trigger slide 60 in a retracted position. With the trigger slide 60 in a retracted position, the arming member 80 is forced towards an extended position shown in FIG. 1 and the trigger member 70 is moved to an initial position.

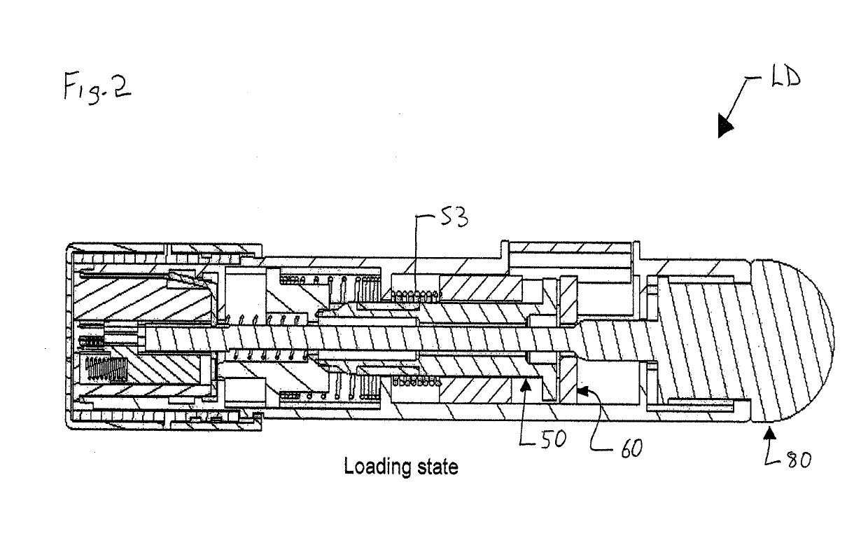

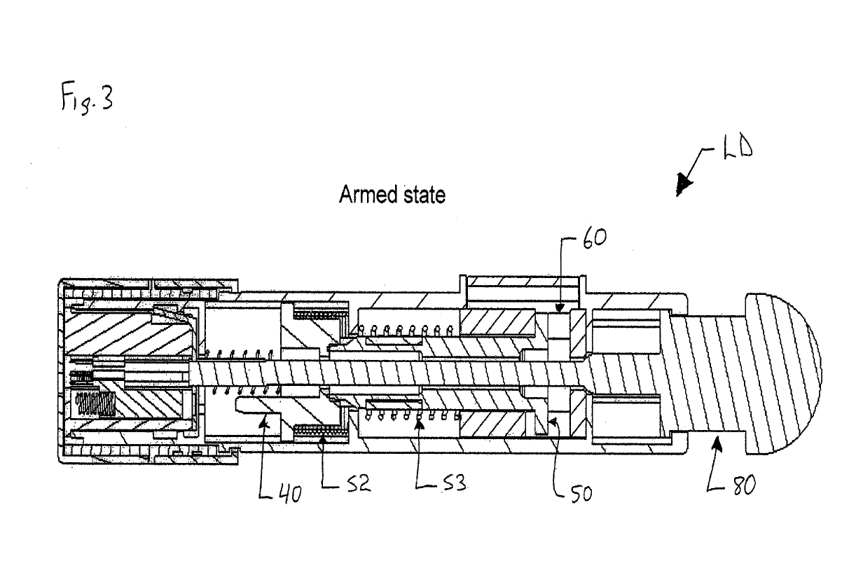

In the position shown in FIG. 2, the lancet device LD is shown in the loading position. This is the position which precedes the arming position shown in FIG. 3. In this position, the return spring S1 is in a slightly compressed position. The drive spring S2 is in a relaxed position and the arming spring S3 is in a substantially fully compressed position owing to the fact that the front surface of the trigger slide member 60 has been forced fowardly by forward movement of the arming member 80. This would occur when a user places a thumb against the outer rounded rear end of the arming member 80 and presses it inwardly relative to the housing 10.

In the position shown in FIG. 3, the lancet device LD is shown in the armed position. This is the position that the device would assume just before the device is triggered or fired. In this position, the return spring S1 is in a relaxed or non-compressed position. The drive spring S2 is in a substantially fully compressed position owing to the fact that an outer shoulder of the front plunger member 40 has been forced rearwardly by rearward movement of the trigger slide 60. The trigger slide 60 was forced rearwardly by the arming spring S3 and caused the rearward movement of the front plunger 40 by locking (during movement from the position shown in FIG. 2 to that shown in FIG. 3) with a rear flange of the rear plunger member 50 (which is connected to the front plunger member 40). The arming spring S3 is again (i.e., just as in FIG. 1) in a slightly compressed position with the arming spring S3 applying a biasing force against the front surface of the trigger slide member 60 which in, in turn, places the trigger slide 60 in a retracted position. With the trigger slide 60 in a retracted position, the arming member 80 is again forced towards an extended position shown in FIG. 3 and the trigger member 70 is again in an initial position.

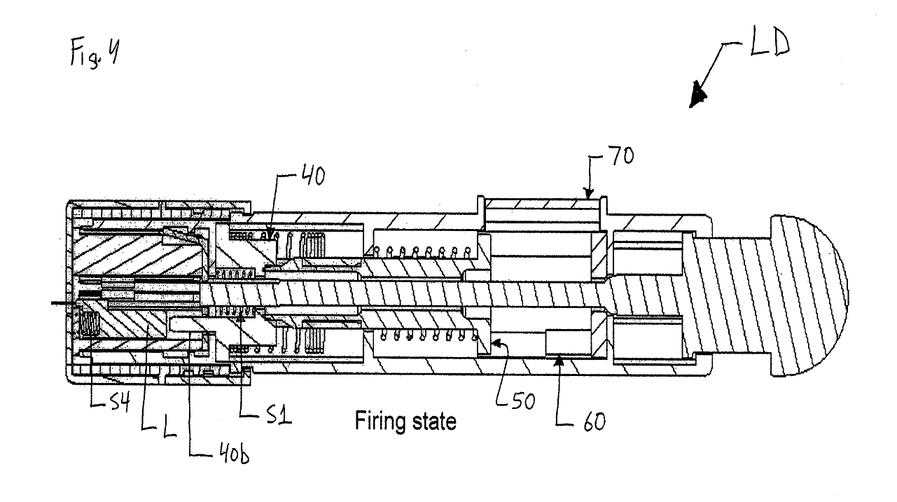

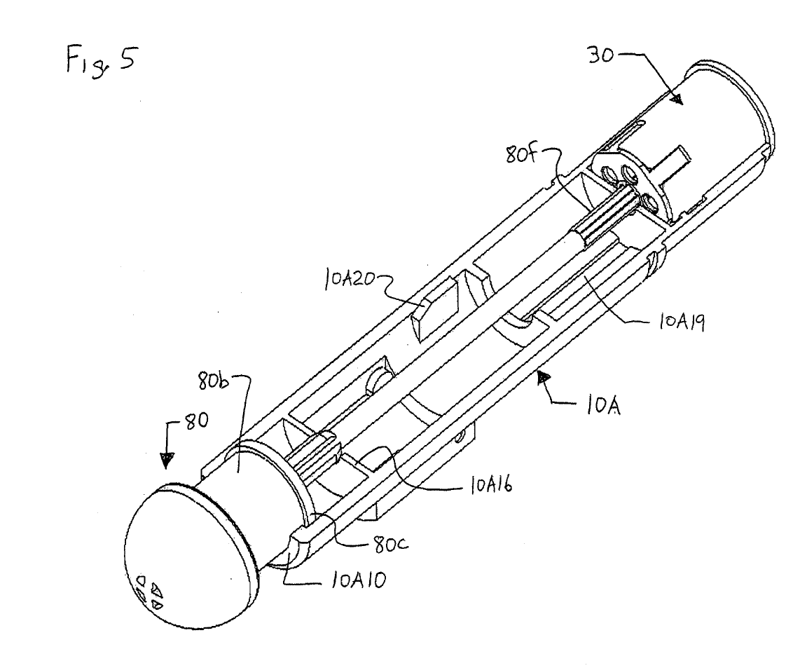

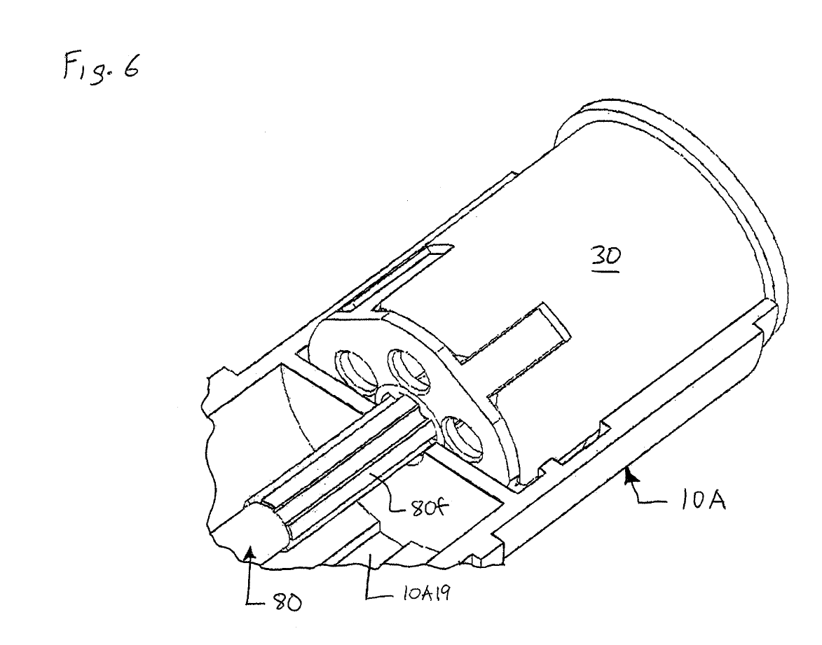

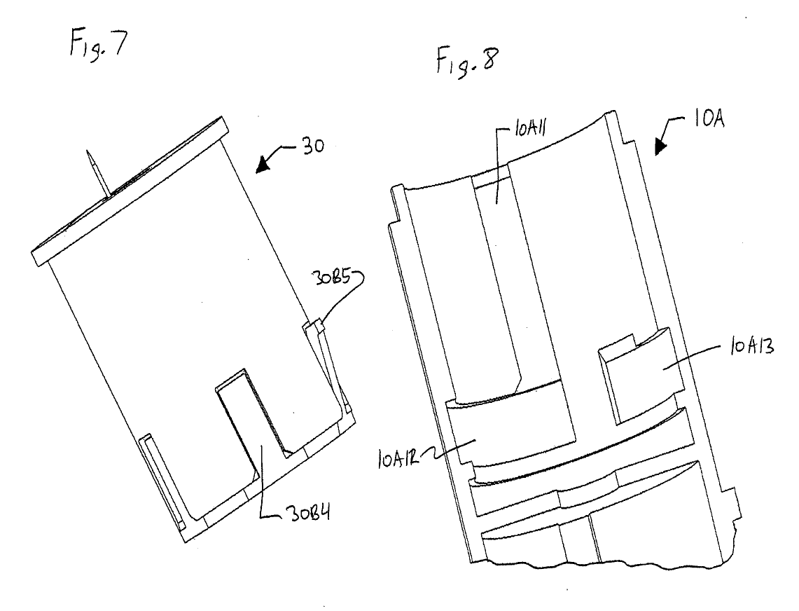

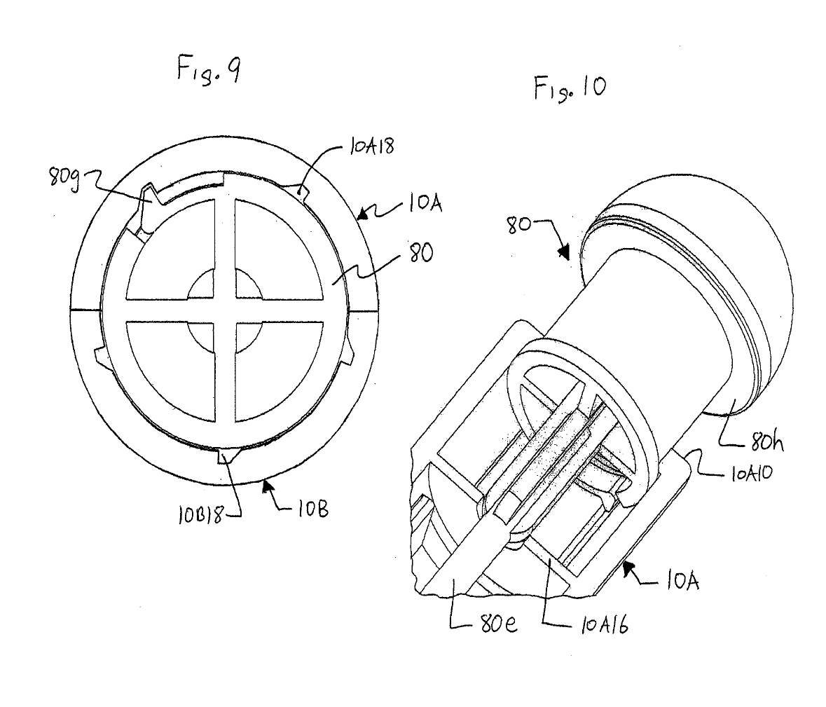

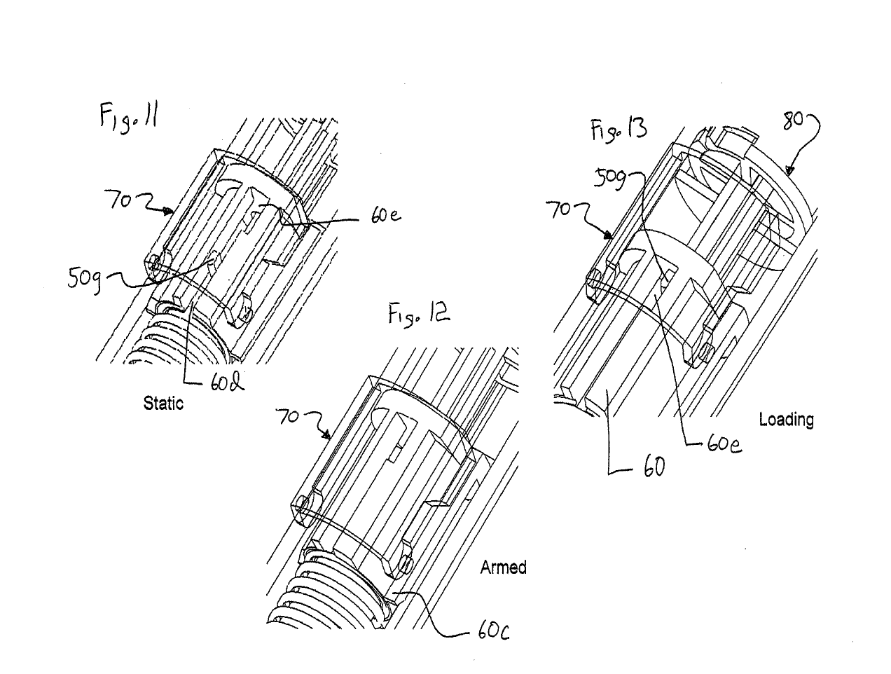

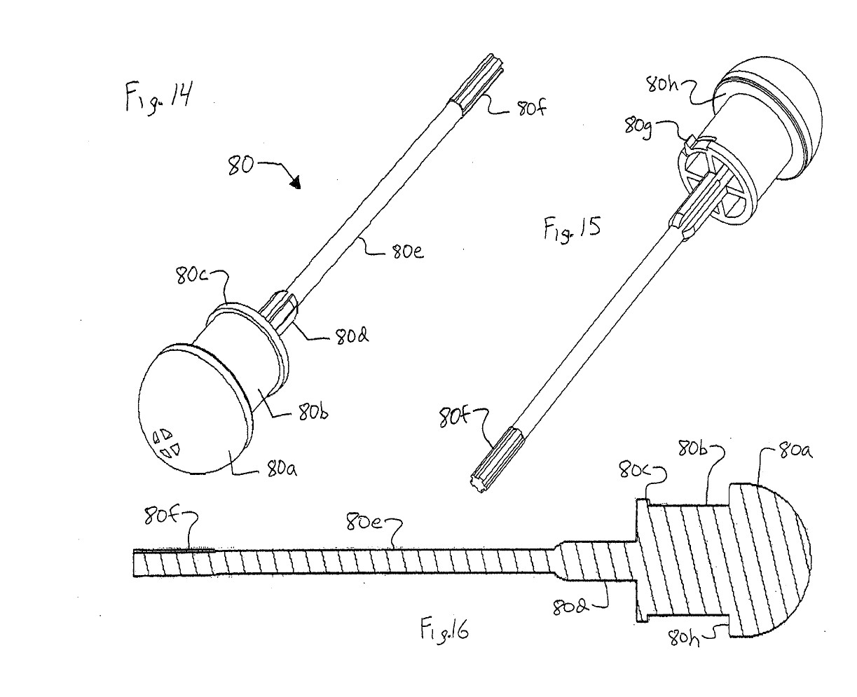

In the position shown in FIG. 4, the lancet device LD is shown in the firing position. This is the position in which a user depresses the trigger 70 thereby automatically causing one of the lancets L in the cartridge 30 to project out of the front surface of the front cap 20 and cause a puncture in a user's skin. Of course, the position shown in FIG. 4 merely shows a snap-shot of the lancet needle extended position. In actuality, the lancet needle would move from the position in FIG. 3 (fully retracted or initial position) to that of FIG. 4 (fully extended or puncturing position), and finally to that of FIG. 1 (fully retracted or initial position) in a fraction of a second. In the firing position , the return spring S1 is in a substantially fully compressed position owing to the forward movement of the members 50/40 as caused by the rapid axial expansion of the drive spring S2 acting on the front flange of member 40. After the members 50/40 move a maximum forward position whereby a pushing member 40b contacts a rear surface of one of the lancets L and moves the lancet L to the puncturing position, the return spring S2, which has substantially reached a maximum amount of compression, will expand back to an original position shown in FIG. 1, which, in turn, places the lancet device LD back in the position shown in FIG. 1. Furthermore, as the return spring S2 expands back to an original position shown in FIG. 1, a lancet return spring S4, which has been substantially fully compressed by forward movement of the lancet L, expands axially to cause the lancet L to move back to an initial position shown in FIGS. 1-3.

The details of the lancet device LD will now be described with reference to FIGS. 1-55. As is apparent from FIGS. 5-11, the lancet device LD utilizes a revolver-type cartridge assembly 30. The cartridge 30 has a generally cylindrical shape and is sized and configured to house a plurality of lancets L. In the exemplary embodiment, the cartridge 30 includes five lancets L. Of course, the cartridge 30 can utilize a few as two, but preferably utilizes any number between 3 and 12 or more. The cartridge assembly 30 is capable of being inserted (rear end first) into an open front end of the housing 10. Insertion takes place by first rotatably aligning a locking member 30B5 of the cartridge 30 with an insertion groove 10A11 of the housing (see FIGS. 7 and 8). Then, the user slides the cartridge 30 into the housing 10 until the locking member 30B5 snaps into the circumferential groove 10A12. The cartridge 30 is now installed on the housing 10 can cannot be removed until it is rotated to a final position whereby the locking member 30B5 is aligned with a tapered surface at the end of the circumferential groove 10A13 (see FIG. 8).

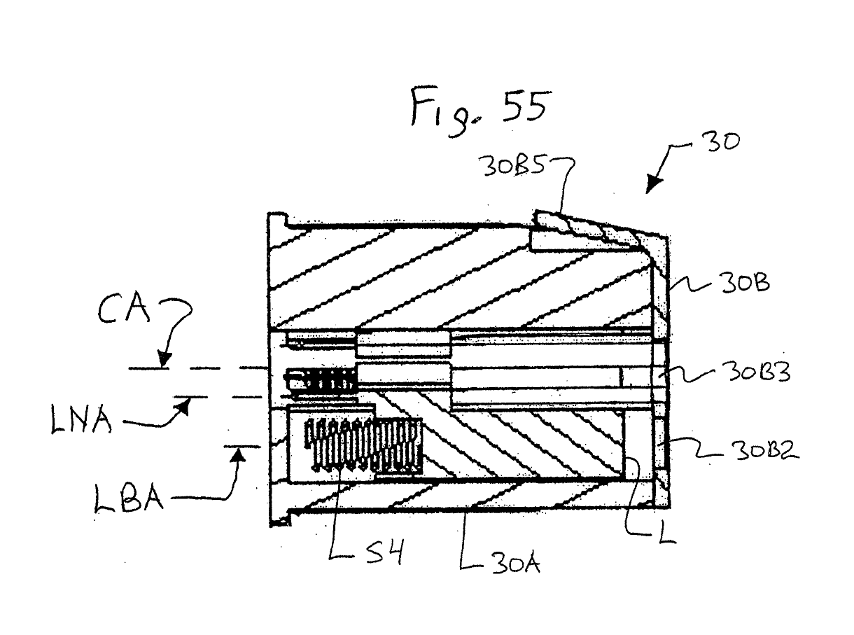

With reference to FIGS. 5, 6, 9 and 10, it can be seen how the installed cartridge 30 can be rotated between discrete positions with each position placing one of the lancets L in a rotational position which aligns the lancet needle LN with an opening 20b in the front cap 20 and which substantially aligns a main axis LBA (see FIG. 55) of one of the lancets L with the pushing member 40b of the front plunger member 40. As is apparent from FIGS. 5 and 6, the cartridge 30 is caused to rotate by the arming member 80 and more specifically by virtue of the fact that that a front portion of the arming member 80 utilizes a spline 80f which engages with a correspondingly shaped recess 30B3 in the cartridge 30. The cartridge 30 is rotationally retained (or temporarily locked) in each of five (i.e., a number corresponding to the number of lancets L in the cartridge 30) discrete angular positions by engagement between a deflecting projection 80g and one of the recesses 10A18 (see FIGS. 9 and 10). As is apparent from FIG. 9, the shape of the deflecting projection 80g and the recesses 10A18 and 10B18 is such that only counterclockwise rotation of the arming member 80 relative to the housing 10 is possible. Of course, one-way rotation can occur in the opposite direction, i.e., clockwise, by reversing the configuration of the projection and recesses. Furthermore, the invention also contemplates other systems for providing one-way rotation such as placing the system shown in FIG. 9 on other areas of the arming member 80 or even on the cartridge 30 itself.

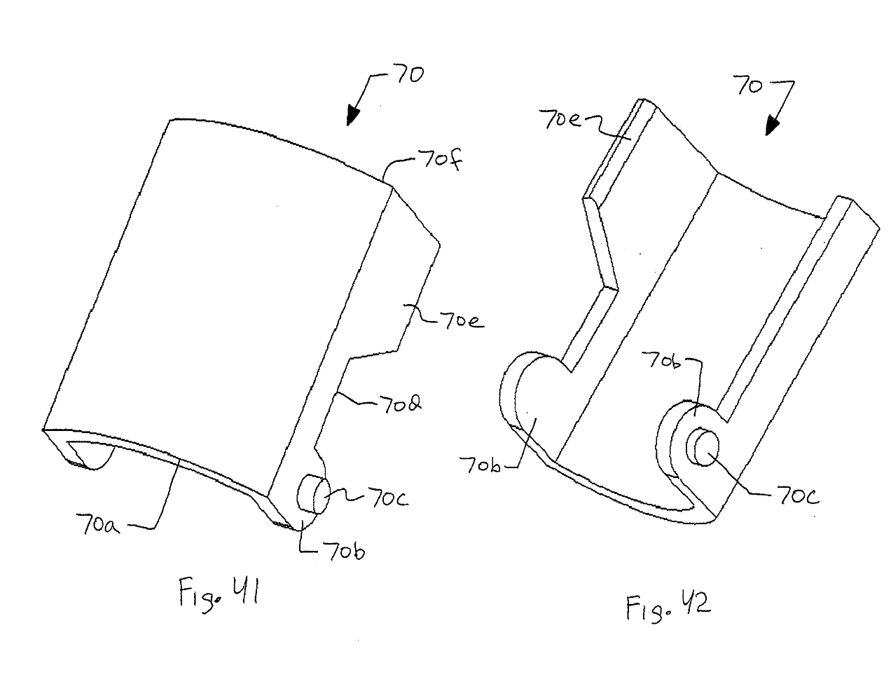

With reference to FIGS. 11-13, it can be seen how the trigger 70, trigger slide 60, and rear plunger member 50 of the lancet device LD are positioned during the static position shown in FIG. 1, during the loading position shown in FIG. 2 and during the arming position shown in FIG. 3. As is evident in FIG. 11, the two opposite facing rear engaging projections 50g (see FIG. 17) are free to move axially within the oppositely arranged slots 60d of the slide member 60. When the arming member 80 is moved forwards as shown in FIG. 2, the slide member 60 is caused to move forward relative to the projections 50g until the retaining recesses 60e move past the projections 50g. At about this point, the front ends of the side portions which have the surfaces 60c contact tapered rear-facing surfaces of the projections 10A20 and 10B20. This causes the slide member 60 to rotate slightly relative to the member 50 thereby moving the projections 50g from alignment with the slots 60d to a position within the recesses 60e (see FIG. 13). Now, when the slide member 60 is caused to move rearwards, the surfaces 60f (see FIG. 23) contact the projections 50g and force the member 50 (as well as member 40 which is fixed thereto) to move rearwards with the slide member 60 until the slide member 60 reaches the position shown in FIGS. 3 and 12. Furthermore, in this position, one of the surfaces 60c engages the projection 70e (see FIGS. 41-42) and places the trigger 70 in an initial or trigger-set position. Thus, when it is desired to trigger the lancet device LD, the user presses inwardly on the trigger 70 thereby causing the projection 70e to apply a downward force of the surface 60c. This, in turn, causes the slide member 60 to rotate to the point wherein the projections 50g leave the recesses 60e and become aligned with the slots 60d. As soon as there is no longer contact between the surfaces 60f and the projections 50g, the drive spring S2 expands axially releasing its energy and causing rapid forward movement of the plunger system 50/40 (see FIG. 4). As a result, the projections 50g move forwards within the slots 60d and are linearly guided thereby.

With reference to FIGS. 14-16, it can be seen that the arming member 80 includes a rounded rear end 80a which serves as a push-button (which can preferably be activated by a user's thumb) to arm the lancet device LD and as a gripping portion which allows the user to rotate the arming member 80 between different angular positions and thereby cause the cartridge assembly 30 to be rotated between different angular positions. Full forward movement of the portion 80a is limited by contact between the annular surface 80h and the annular surface formed by semi-annular surfaces 10A10 and 10B10 (see 37-40). Arranged inwardly or forwardly from the portion 80a is a generally cylindrical surface 80b which is sized to slidably engage (i.e., axially and rotatably) with a generally cylindrical surface formed by semi-circular surfaces 10A17 and 10B17 of the housing 10. Arranged inwardly from the surface 80b is a generally circular projection or shoulder 80c which is sized to slide within (i.e., axially and rotatably) a generally cylindrical open area defined between by housing projections 10A16 and 10B16 and the housing projections having semi-circular surfaces 10A17 and 10B17 of the housing 10. A deflecting projection 80g is integrally formed on the flange 80c. This projection 80g is configured to selectively engage with and disengage with a plurality of grooves 10A18 and 10B18 (see FIGS. 9 and 10). The grooves 10A18 and 10B18 remain engage with the projection 80g throughout an axial length of travel of the member 80. Arranged inwardly from the shoulder 80c is a spline shaped cylindrical portion 80d which is sized to slide within (i.e., axially and rotatably) a generally circular-shaped opening defined by housing projections 10A16 and 10B16. Arranged inwardly from the projection 80d is a generally cylindrical-shaped shaft portion 80e which is sized to slide within (i.e., axially and rotatably) members 60, 50 and 40. Arranged inwardly from the shaft 80e is a spline portion 80f which is sized to slide within a spline-shaped opening 30B3 and generally cylindrically-shaped opening 30A2 (see FIG. 28). In particular, the spline 80f engages with the opening 30B3 in a rotatably fixed manner (i.e., rotation of the spline 80f causes rotation of the cartridge 30) while also allowing the spline 80f to slide axially within the openings 30B3 and 30A2. As is apparent from FIGS. 14-16, the arming member 80 can preferably be a one-piece member and is most preferably a one-piece synthetic resin member. Of course, the member 80 can also be an assembly of plural components provided it functions in a manner similar to that of the member shown in FIGS. 14-16.

FIGS. 17 and 18 show a plunger assembly used in the lancet device LD. The plunger assembly utilizes a forward or front plunger member 40 and a rear plunger member 50. An arming spring S3 is placed around the member 50 before the members 40 and 50 become axially connected to each other via the recesses 40g and projections 50d. The plunger assembly functions as discussed above in reference to FIGS. 1-4.

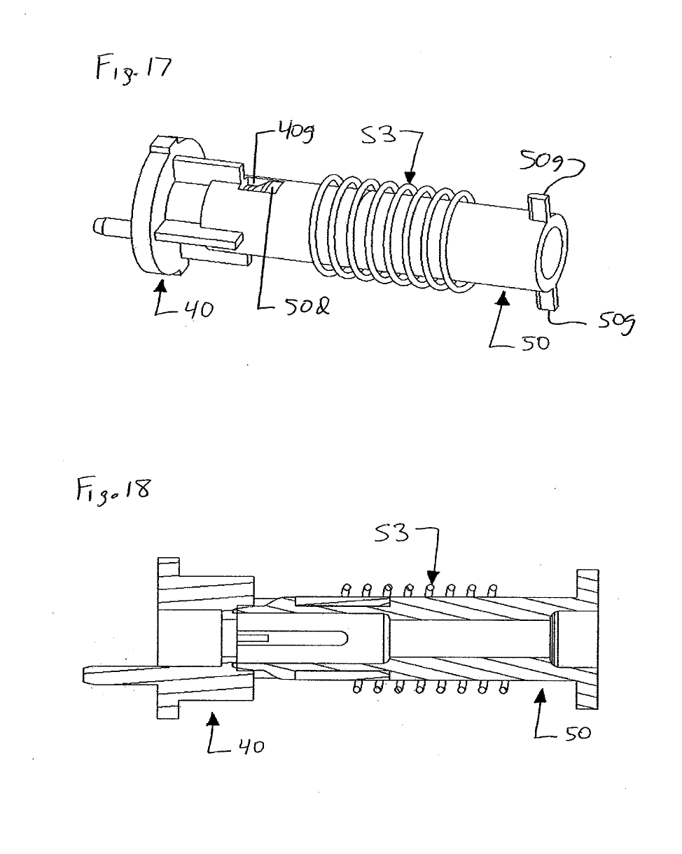

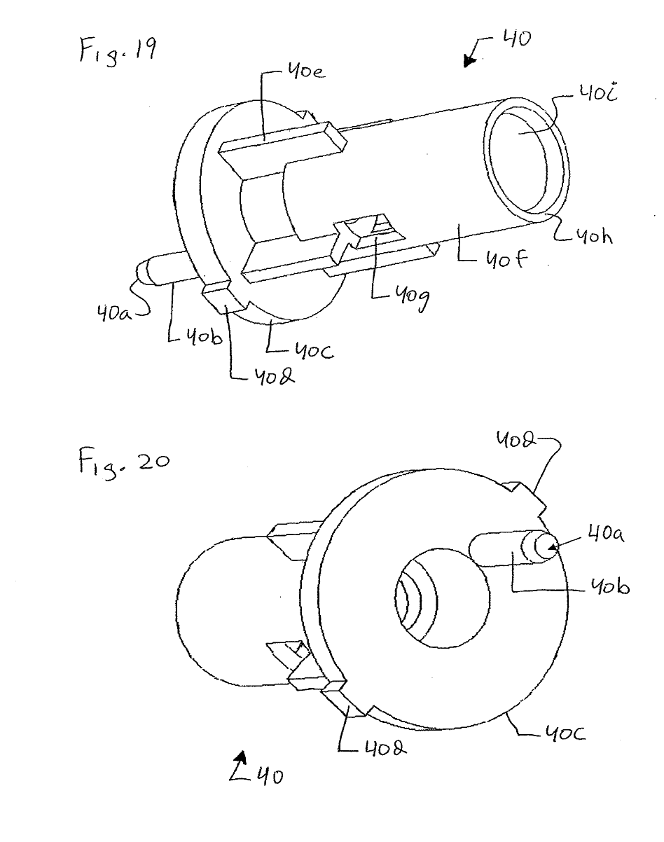

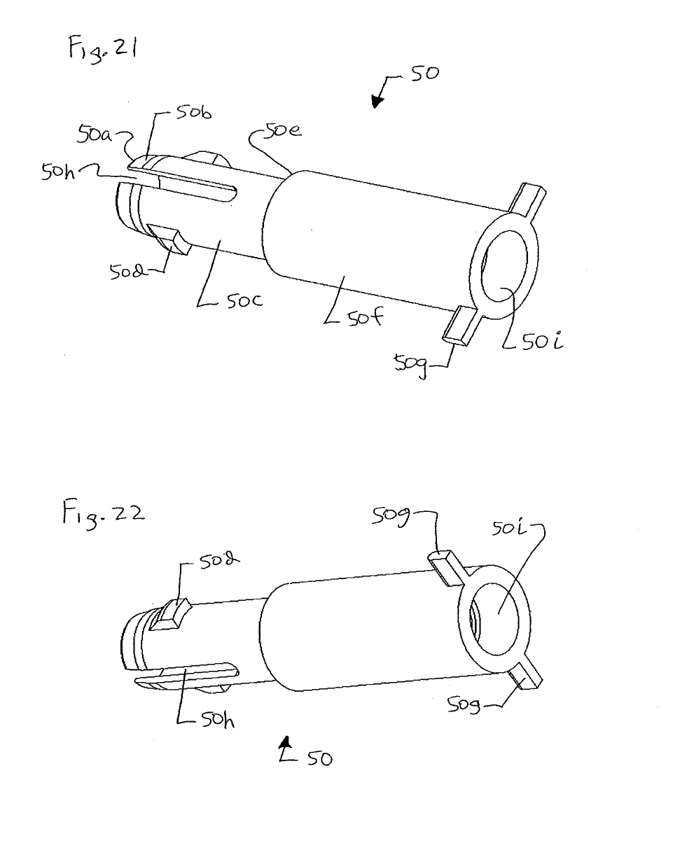

With reference to FIGS. 19 and 20, it can be seen that the forward plunger member 40 includes a forward facing generally cylindrical pushing member 40b having a front end surface 40a which is structured and arranged to contact a rear surface of a lancet L when the lancet device LD is fired (see FIG. 4). Arranged inwardly or rearwardly from the portion 40b is a generally circular flange 40c which is sized to slide within a generally spaced defined by semi-circular projections 10A14 and 10B14 and 10A15 and 10B15 of the housing 10. Two oppositely arranged projections 40d are arranged on the flange 40c. One of the projections 40d slidably engages and is linearly guided by a guide groove 10A19 (see FIG. 38) in upper housing part 10A and another of the projections 40d slidably engages and is linearly guided by a guide groove 10B19 (see FIG. 40) in lower housing part 10B. Four radially oriented elongated projections 40e extend to the flange 40c. These projections 40e serve to support a front portion of the drive spring S2 (see FIGS. 1-4). Two oppositely arranged recesses 40g are arranged on a generally cylindrical portion 40f. Once a front end of the member 50 is fully inserted (defined by contact between annular surface 40h and 50e) into a rear opening 40i of the member 40, one of the recesses 40g lockingly engages with one of the projections 50d (see FIGS. 21 and 22) in the rear plunger member 50 and another of the recesses 40g lockingly engages with another of the projections 50d (see FIGS. 21 and 22) in the rear plunger member 50. As was shown in FIGS. 17 and 18, this assembly procedure preferably occurs after the arming spring S3 is slid or otherwise installed on the member 50. As is apparent from FIGS. 19 and 20, the member 40 can preferably be a one-piece member and is most preferably a one-piece synthetic resin member. Of course, the member 40 can also be an assembly of plural components provided it functions in a manner similar to that of the member shown in FIGS. 19-20.

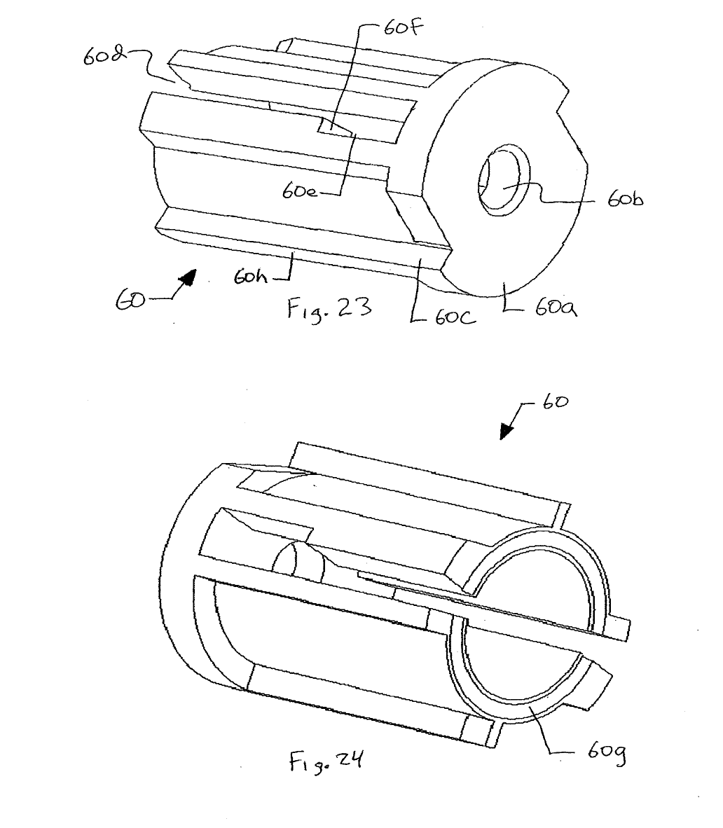

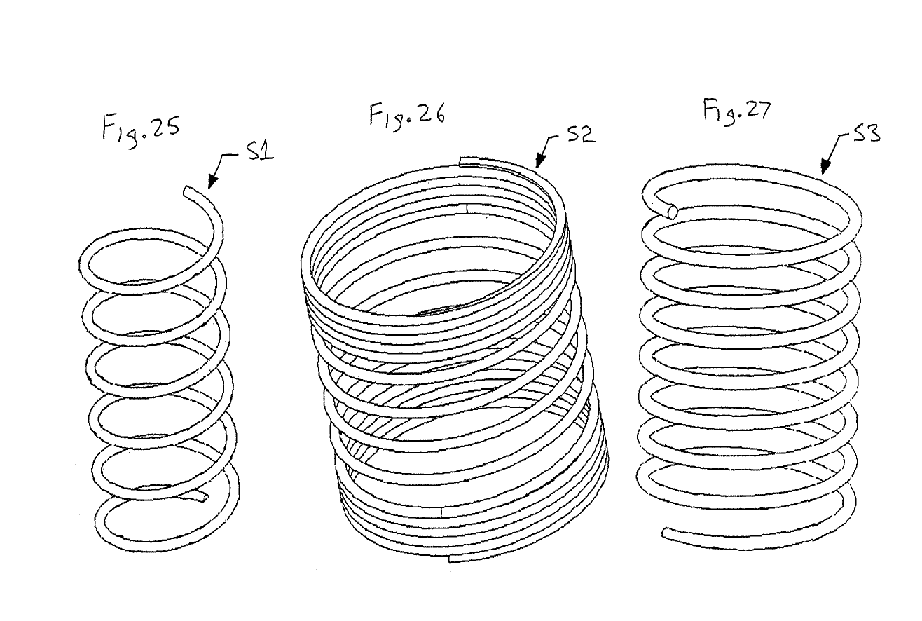

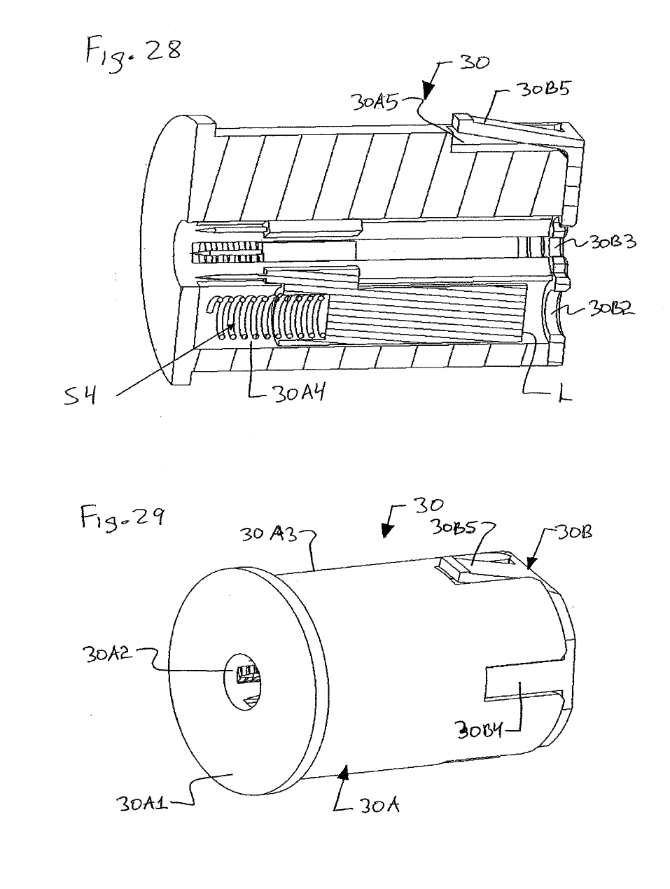

With reference to FIGS. 21 and 22, it can be seen that the rear plunger member 50 includes a front end 50a. A tapered portion 50b extends to a generally cylindrical portion 50c and facilitates insertion of the portion 50c into the opening 40i. An annular shoulder 50e is configured to contact annular end 40h. Two oppositely arranged projections 50d are arranged on the portion 50c. One of the projections 50d lockingly engages with one of the recesses 40g in the front plunger member 40 and another of the projections 50d lockingly engages with another of the recesses 40g in the front plunger member 40. As was shown in FIGS. 17 and 18, this assembly procedure preferably occurs after the arming spring S3 is slid or otherwise installed on the member 50. Two oppositely arranged projections 50g are arranged on a back end of a generally cylindrical portion 50f. One of the projections 50g is configured to slide within one of the slots 60d of member 60 (see FIGS. 23-24) and to lockingly engage with one of the recesses 60e in the slide member 60. Another of the projections 50g is similarly configured to slide within another of the slots 60d of member 60 and to lockingly engage with another of the recesses 60e in the member 60 (see also discussion above with regard to FIGS. 11-13). An opening 50i (as well as opening 40i) is sized to allow the passage therethrough of the shaft portion 80e of the arming member 80 (see FIGS. 1-4). As is apparent from FIGS. 21 and 22, the member 50 can preferably be a one-piece member and is most preferably a one-piece synthetic resin member. Of course, the member 50 can also be an assembly of plural components provided it functions in a manner similar to that of the member shown in FIGS. 19-20.

With reference to FIGS. 23 and 24, it can be seen that the trigger slide member 60 includes a rear end 60a and a front end 60g. Two generally oppositely arranged ribs 60h are arranged on the member 60. One of the ribs 60h includes an upward facing surface 60c which is configured to engage and be contacted by the projection 70e of the trigger 70 (see FIGS. 41-42). Two oppositely arranged slots 60d are open an a front end of the member 60 and extend to locking recesses 60e. As explained above, one f the projections 50g of the member 50 is configured to slide within one of the slots 60d of member 60 and to lockingly engage with one of the recesses 60e in the slide member 60. Another of the projections 50g is similarly configured to slide within another of the slots 60d of member 60 and to lockingly engage with another of the recesses 60e in the member 60 (see also discussion above with regard to FIGS. 11-13). An opening 60b is sized to allow the passage therethrough of the shaft portion 80e of the arming member 80 while also preventing insertion of the portion 80d of the member 80 (see FIGS. 1-4). Thus, when the portion 80d contacts the opening 60b, the member 60 is caused to move axially with a generally cylindrical space defined by projections 10A16 and 10B16 and 10A15 and 10B15. Of course, the member 60 moves axially forwards only up to the point (see FIG. 2) that it makes contact with projections 10A20 and 10B20, with this movement being limited by contact between surfaces 80h and 10A10 (see FIG. 10). As is apparent from FIGS. 23 and 24, the member 60 can preferably be a one-piece member and is most preferably a one-piece synthetic resin member. Of course, the member 60 can also be an assembly of plural components provided it functions in a manner similar to that of the member shown in FIGS. 23-24.

With reference to FIGS. 25-27, it can be seen that the return spring S1, the drive spring S2 and the arming spring S3 can have the form of helical wire compression springs. Each spring S1-S3 is preferably be a one-piece member and is most preferably a one-piece spring metal member. Of course, the springs can also be made of any material provided they function in a manner similar to that of the member shown in FIGS. 25-27. The arming spring S3 preferably withstands greater compressive forces than the drive spring S2. The drive spring S2 preferably withstands greater compressive forces than the return spring S1.

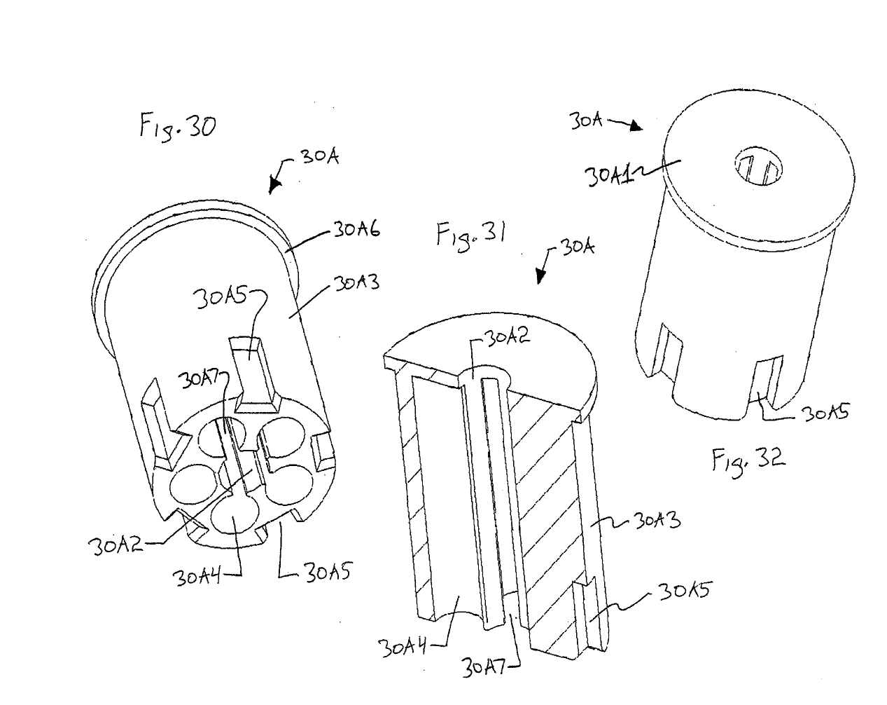

With reference to FIGS. 28-36, it can be seen that the cartridge assembly 30 includes a cartridge body 30A, a cartridge retainer 30B, lancets L, and lancet return springs S4. The cartridge body 30A has front surface 30A1 which defines a centrally disposed generally circular opening 30A2. The opening 30A2 is sized and configured to allow the lancet needles LN of the lancets L to project out past the surface 30A1. Arranged inwardly or rearwardly from the surface 30A1 is a generally annular shoulder 30A6 which is sized to substantially abut an annular front surface defined by semi-circular surfaces 10A1 and 10B1 of the housing 10 when the locking member 30B5 snaps into the circumferential groove 10A12 (see FIGS. 7 and 8). Arranged inwardly or rearwardly from the shoulder 30A6 is a generally cylindrical surface 30A3 which is sized to slide (i.e., axially upon insertion and removal and rotationally when fully installed in the housing 10) within a generally cylindrical open cylindrical space formed by semi-cylindrical surfaces 10A21 and 10B21 of the housing 10. Five generally rectangular-shaped recesses 30A5 are substantially equally angularly spaced and are arranged a rear end of the body 30A. These recesses 30A5 are configured to receive therein four tab portions 30B4 (see FIGS. 35 and 36) of the cartridge retainer member 30B and a locking tab 30B5 which, as discussed above, allows the cartridge assembly 30 to slide into the front of the housing 10 at only one rotational position, to be removed at a different rotational position, and to become locked within circumferential groove defined by groove portions 10A12, 10B12, and 10A13. Five substantially equally spaced cylindrical openings 30A4 are arranged in the body 30A. Each opening 30A4 is sized to slidably receive therein one of the lancets L and includes a guide slot 30A7 which ensures that the lancets L are substantially linearly guided (i.e., without substantially rotating) and which allows the lancet needle portion LN of the lancets L to move within the central opening 30A2. As is apparent from FIGS. 30-32, the member 30A can preferably be a one-piece member and is most preferably a one-piece synthetic resin member. Of course, the member 30A can also be an assembly of plural components provided it functions in a manner similar to that of the member shown in FIGS. 30-32.

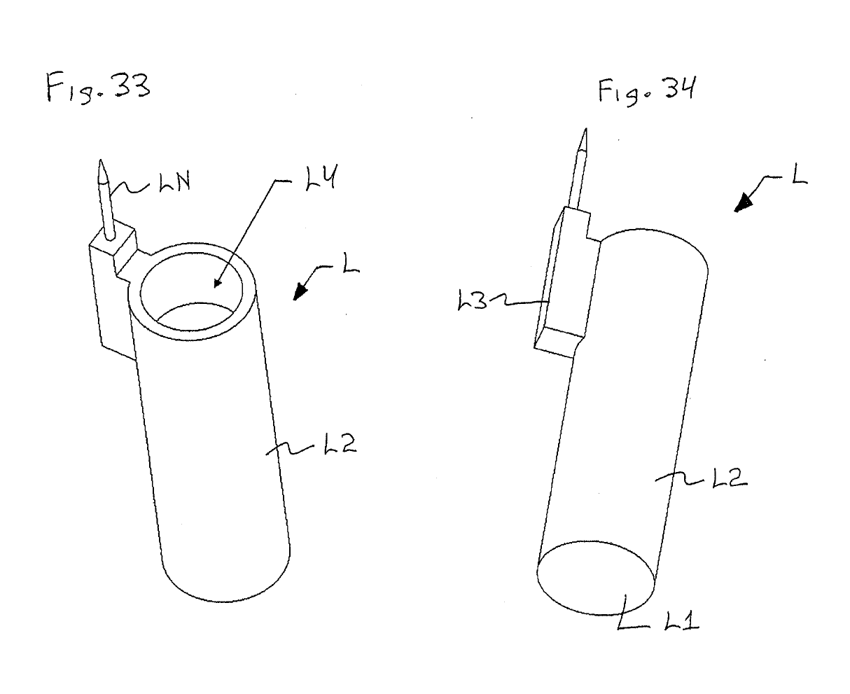

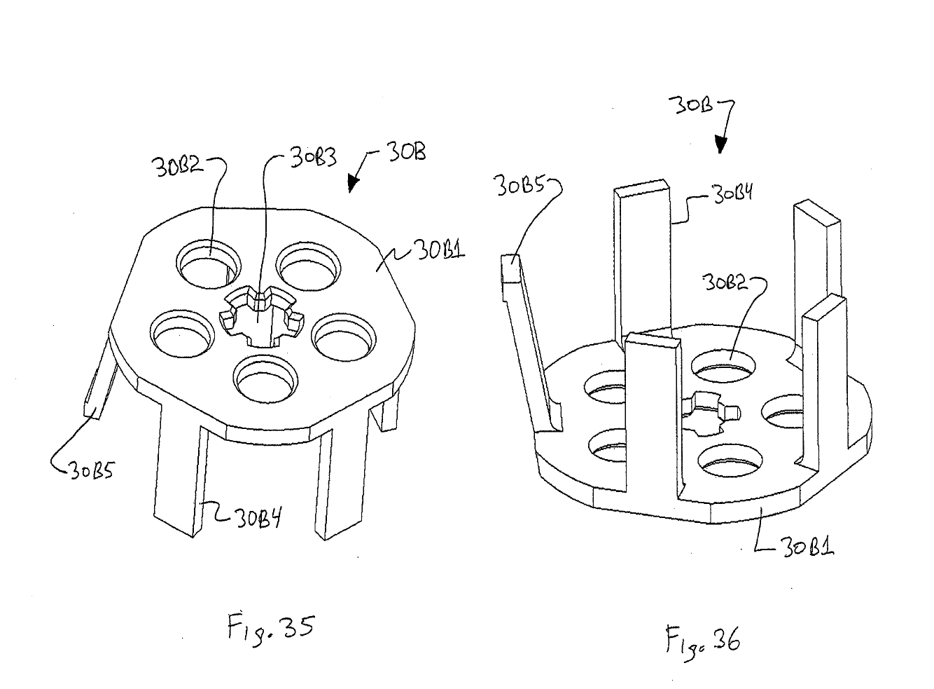

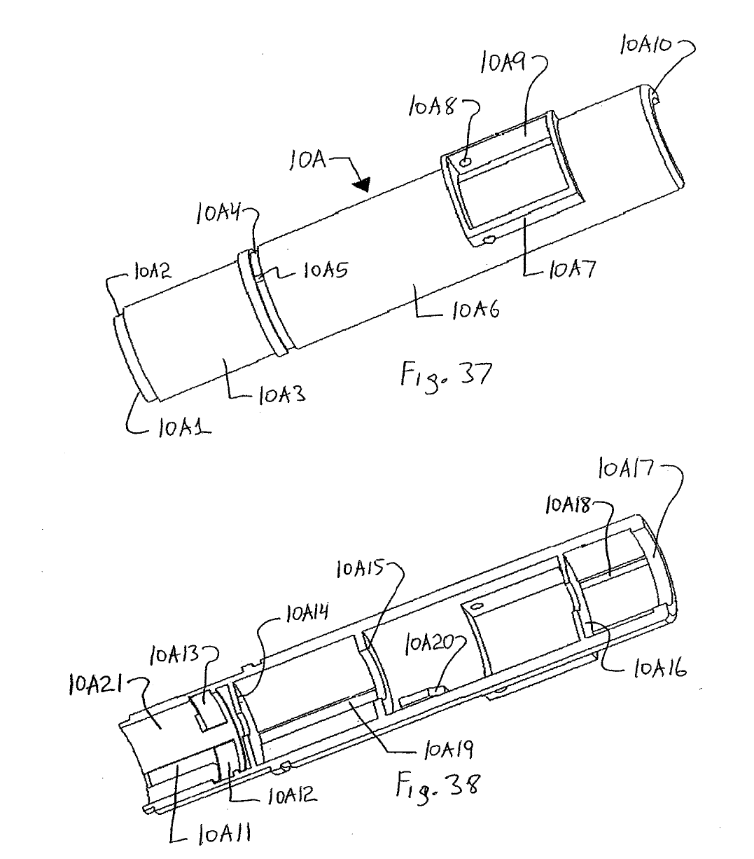

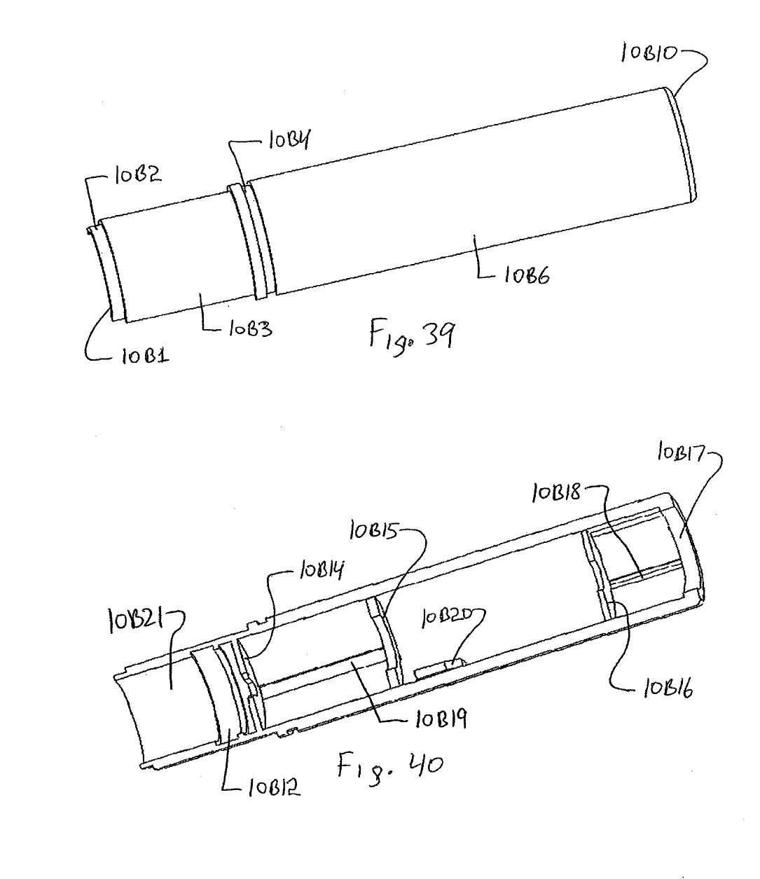

With reference to FIGS. 33-34, it can be seen that each lancet L includes a lancet needle LN, a rear surface L1, a main body portion L2, a lancet needle support portion L3, and a open front end L4. The lancet needle LN can be of any size typically used for lancet needles and preferably has a sharp free end which is configured to puncture a user's skin while causing minimal pain. The rear surface L1 is generally circular and is configured to be contacted by the end surface 40a of the front plunger member 40 when the lancet device LD is fired (see FIG. 4). The main body portion L2 is generally cylindrical and is sized and configured to freely slide within opening 30A4 of the cartridge body 30A. The lancet needle support portion L3 has a generally rectangular shape whose opposite planar surfaces are sized and configured to freely slidably engage with corresponding side surfaces of forming the groove 30A7. The open front end L4 is a generally cylindrical blind opening which is sized and configured to receive therein a rear portion of the lancet return spring S4. As is apparent from FIGS. 33-34, the lancets L can preferably be a one-piece member (with the exception of the lancet needle LN) and is most preferably a one-piece synthetic resin member. Of course, the lancets L can also be an assembly of plural components provided it functions in a manner similar to that of the member shown in FIGS. 33-34.

With reference to FIGS. 35-36, it can be seen that the cartridge retainer 30B includes a generally planar base portion 30B1, five equally angularly spaced projecting tab portions 30B4/30B5, a plurality of equally angularly spaced openings 30B2, and a centrally arranged opening 30B3 having an internal spline which is sized to engage with the spline portion 80f of the arming member 80. The four tab portions 30B4 are oriented generally perpendicular to the base portion 30B1 and are configured to frictionally engage with the corresponding four recesses 30A5 of the cartridge body 30A so as to secure the cartridge retainer 30B to a rear end of the cartridge body 30A as shown in FIG. 28. The openings 30B2 are generally circular and are sized and configured to be aligned with the openings 30A4 of the cartridge body 30A and allow for the passage therethrough of the pushing portion 40b of the front plunger member 40 and allows the surface 40a to contact the rear surface L1 of the lancet L when the lancet L is position in the appropriate position for firing. A locking tab 30B5 does not frictionally engage with a corresponding recess 30A5 of the cartridge body 30A, but is capable of deflecting inwardly into the recess 30A5 upon insertion and removal of the cartridge assembly 30 from the hosing 10. As discussed above, the locking tab or member 30B5 allow the cartridge assembly 30 to slide into the front of the housing 10 at only one rotational position (i.e., when aligned with groove 10A11), to be removed at a different rotational position (i.e., at the end of groove 10A13), and to become locked within circumferential groove defined by groove portions 10A12, 10B12, and 10A13. As is apparent from FIGS. 35-36, the cartridge retainer 30B can preferably be a one-piece member and is most preferably a one-piece spring metal member. Of course, the member 30B can also be an assembly of plural components provided it functions in a manner similar to that of the member shown in FIGS. 35-36.

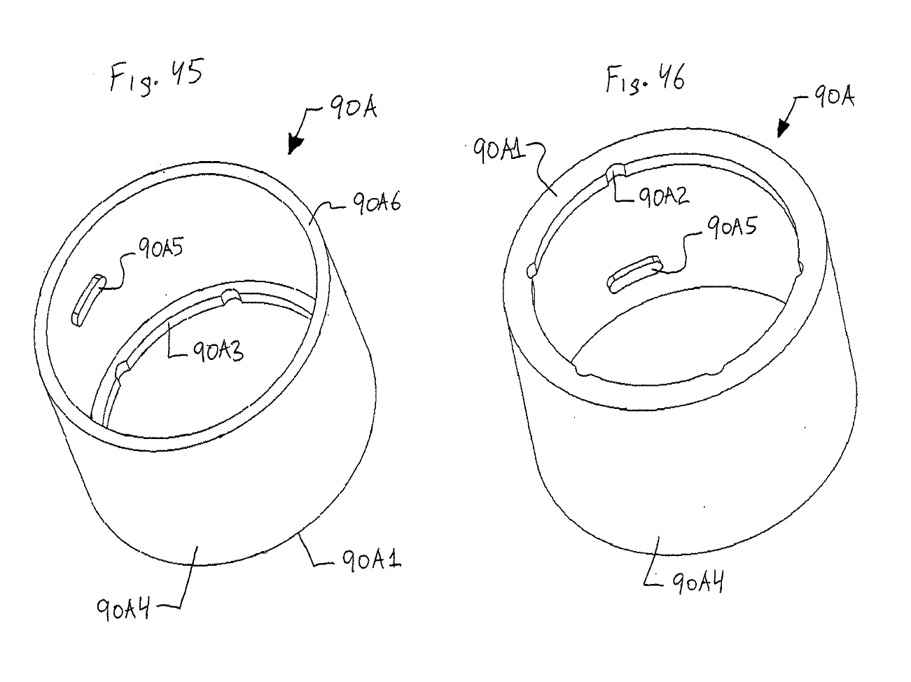

With reference to FIGS. 37-40, it can be seen that the housing 10 preferably be a two-piece housing formed by an upper housing member 10A and a lower housing member 10B. These parts 10A and 10B can be connected together to form housing 10 by any convenient mechanisms such as snap-connection, projections and recesses, adhesive bonding, ultrasonic welding, etc. The upper housing part 10A includes a front semi-annular surface 10A1, a generally semi-circular circumferential recess 10A2, a generally semi-cylindrical front portion 10A3, a semi-circular circumferential recess 10A4 which includes a projection 10A5 and which is sized to receive therein the circular flange 90A3 of the outer sleeve 90 (see FIGS. 45 and 46), a semi-cylindrical main surface 10A6 which includes a generally rectangularly-shaped trigger opening 10A9 defined by projecting walls 10A7. Two oppositely arranged openings 10A8 are arranged on two of the walls and are sized to receive therein generally circular projections 70c of the trigger member 70 (see FIGS. 41-42). The openings 10A8 and projections 70c allow the trigger member 70 pivot in the opening 10A9. The upper housing part 10A also includes a rear semi-annular surface 10A10, a generally semi-cylindrical surface 10A21, an axially oriented guide groove 10A11, a semi-circular circumferential groove 10A12 which includes a groove end area 10A12, a semi-circular projection or web 10A14 which includes a generally semi-circular opening which allows for the free movement of the spline portion 80f of the arming member 80. A generally cylindrical space is arranged between projection 10A14 and projection 10A15 which also includes a semi-circular opening sized to allow a free movement of the plunger assembly shown in FIGS. 17 and 18. An axially oriented guide groove 10A19 is arranged in this space and serves to slidably receive therein (and linearly guide) one of the projections 40d of the front plunger member 40. Another generally cylindrical space is arranged between projection 10A15 and projection 10A16 which also includes a semi-circular opening sized to allow a free movement of the portion 80d of the arming member 80. An elongated projection 10A20 has a rear facing tapered portion and is sized and configured to be engaged by a tapered front end portion of one of the portions 60h of the slide member 60 in order to cause slight rotation of the member 60 (upon such engagement) when the member 60 is axially moved to the loading position shown in FIG. 2. Another generally cylindrical space is arranged between projection 10A16 and shoulder 10A17 and includes generally equally spaced axially oriented grooves 10A18 which are configured to receive therein the deflecting projection 80g of the arming member 80. As is apparent from FIGS. 37-38, the upper housing part 10A can preferably be a one-piece member and is most preferably a one-piece synthetic resin member. Of course, the member 10A can also be an assembly of plural components provided it functions in a manner similar to that of the member shown in FIGS. 37-38.

With reference to FIGS. 39-40, it can be seen that the lower housing part 10B includes a front semi-annular surface 10B1, a generally semi-circular circumferential recess 10B2, a generally semi-cylindrical front portion 10B3, a semi-circular circumferential recess 10B4 and which is sized to receive therein the circular flange 90A3 of the outer sleeve 90 (see FIGS. 45 and 46), a semi-cylindrical main surface 10B6. The lower housing part 10B also includes a rear semi-annular surface 10B10, a generally semi-cylindrical surface 10B21, a semi-circular circumferential groove 10B12, a semi-circular projection or web 10B14 which includes a generally semi-circular opening which allows for the free movement of the spline portion 80f of the arming member 80. A generally cylindrical space is arranged between projection 10B14 and projection 10B15 which also includes a semi-circular opening sized to allow a free movement of the plunger assembly shown in FIGS. 17 and 18. An axially oriented guide groove 10B19 is arranged in this space and serves to slidably receive therein (and linearly guide) one of the projections 40d of the front plunger member 40. Another generally cylindrical space is arranged between projection 10B15 and projection 10B16 which also includes a semi-circular opening sized to allow a free movement of the portion 80d of the arming member 80. An elongated projection 10B20 has a rear facing tapered portion and is sized and configured to be engaged by a tapered front end portion of one of the portions 60h of the slide member 60 in order to cause slight rotation of the member 60 (upon such engagement) when the member 60 is axially moved to the loading position shown in FIG. 2. Another generally cylindrical space is arranged between projection 10B16 and shoulder 10B17 and includes generally equally spaced axially oriented grooves 10B18 which are configured to receive therein the deflecting projection 80g of the arming member 80. As is apparent from FIGS. 39-40, the lower housing part 10B can preferably be a one-piece member and is most preferably a one-piece synthetic resin member. Of course, the member 10B can also be an assembly of plural components provided it functions in a manner similar to that of the member shown in FIGS. 39-40.

With reference to FIGS. 41-42, it can be seen that the trigger member 70 includes a front end 70a, two generally parallel side walls which each include a projecting portion 70b having an opposite facing circular projection 70c, and a rear end 70f. One of the side walls includes a recess 70d and a projecting portion 70e which is sized and configured to contact the surface 60c of the member 60 and cause the member 60 to rotate when the lancet device LD is to be fired by the user depressing the trigger member 70. As is apparent from FIGS. 41-42, the trigger member 70 can preferably be a one-piece member and is most preferably a one-piece synthetic resin member. Of course, the member 70 can also be an assembly of plural components provided it functions in a manner similar to that of the member shown in FIGS. 41-42.

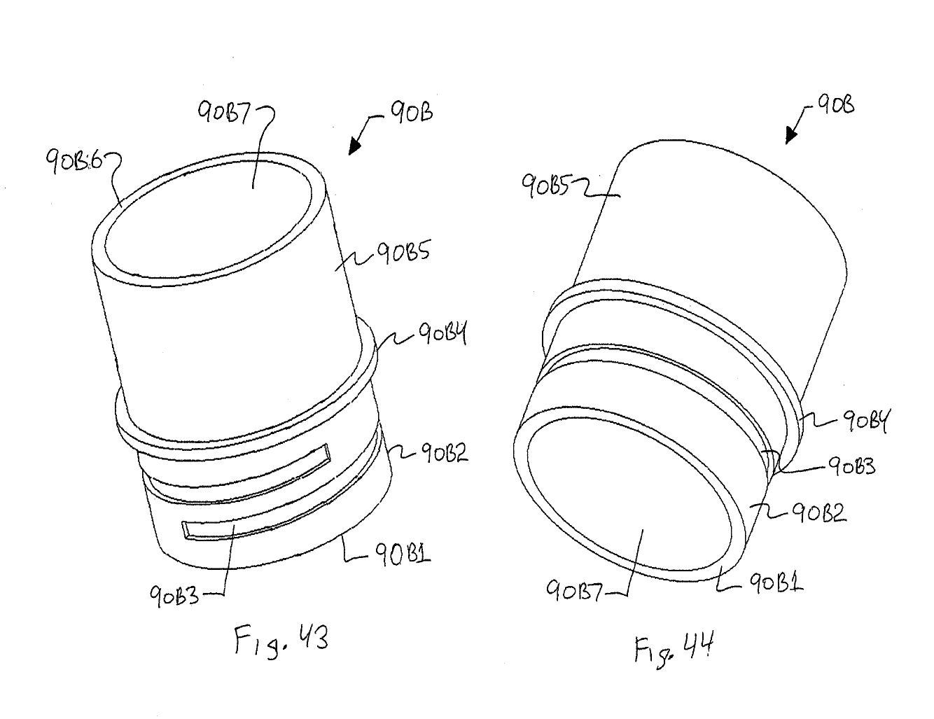

With reference to FIGS. 43-46 and 49, it can be seen that a depth adjustment system 90 includes an inner sleeve 90B and an outer sleeve 90A. The inner sleeve 90B includes a rear annular end 90B1, a generally cylindrical main opening 90B7, a generally cylindrical surface 90B2 that includes a helical groove 90B3 which is sized and configured to slidably receive therein two oppositely arranged projections 90A5 of the outer sleeve 90A. In this way, when the outer sleeve 90A is rotated relative to the housing 10, the interaction of the projections 90A5 and the groove 90B3 causes an axial movement of the inner sleeve 90B relative to the housing 10 and outer sleeve 90A. That is, when the outer sleeve 90A is rotated in one direction relative to the housing 10, the interaction of the projections 90A5 and the groove 90B3 causes an axial movement of the inner sleeve 90B relative to the housing 10 and outer sleeve 90A in one direction and in an opposite axial direction when the outer sleeve 90A is rotated in an opposite direction relative to the housing 10. The depth adjustment inner sleeve 90B also includes a generally ring-shaped flange 90B4 which is configured to abut an annular surface 90A6 of the outer sleeve 90A in an inner most axial position of the inner sleeve 90B and is configured to abut an annular surface 20d of the front cap 20 when the front cap 20 is fully installed on the generally cylindrical surface 90B5. As is apparent from FIGS. 43-44, the member 90B can preferably be a one-piece member and is most preferably a one-piece synthetic resin member. Of course, the member 90B can also be an assembly of plural components provided it functions in a manner similar to that of the member shown in FIGS. 43-44.

With reference to FIGS. 45-46, it can be seen that a depth adjustment outer sleeve 90A includes a rear annular end 90A1, an outer generally cylindrical surface 90A4, and a generally circular inner flange 90A3 which is sized and configured to slidably engage and rotate in a circumferential groove 10A4/10B4 of the housing 10. A plurality of recesses 90A2 are arranged on the flange 90A3 and are sized to receive therein the projection 10A5 in order to temporarily retain the outer sleeve 90A in each of five rotational positions, i.e., one position for each recess 90A2, relative to the housing 10. Two oppositely arranged projections 90A5 (which are axially offset in order that both projections extend into the helical groove 90B3) are arranged on an inner generally cylindrical surface of the outer sleeve 90A. Although not shown, the outer surface 90A4 can include indicia which, when compared to an indicator on the housing, can convey to the user a depth adjustment position of the inner sleeve 90B. As is apparent from FIGS. 45-46, the member 90A can preferably be a one-piece member and is most preferably a one-piece synthetic resin member. Of course, the member 90A can also be an assembly of plural components provided it functions in a manner similar to that of the member shown in FIGS. 45-46.

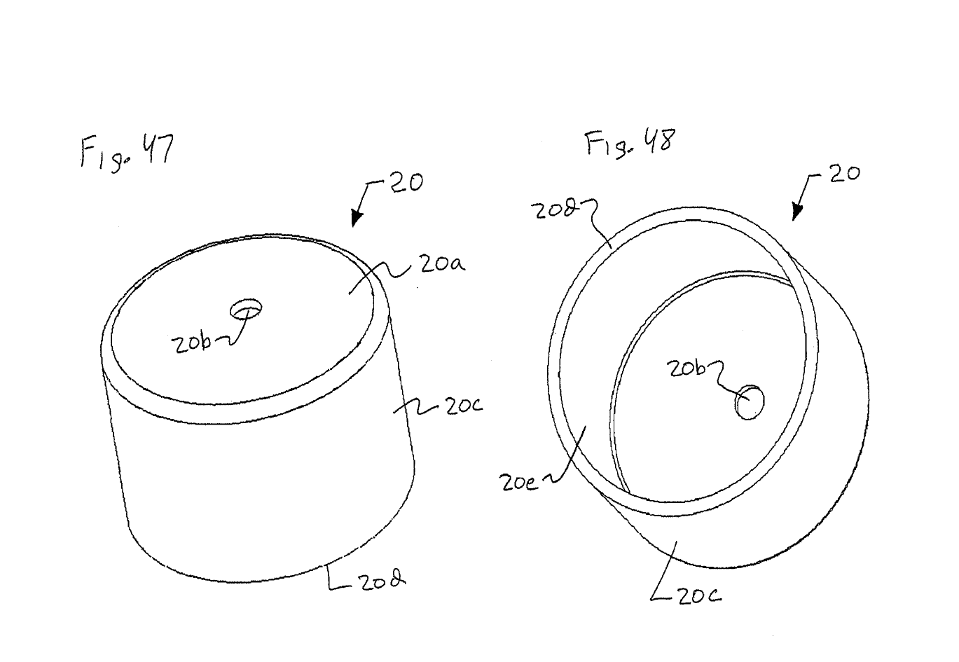

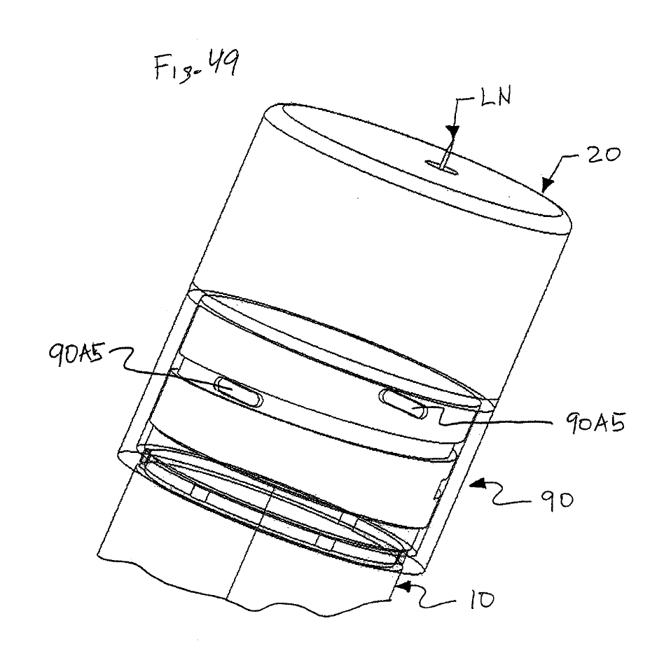

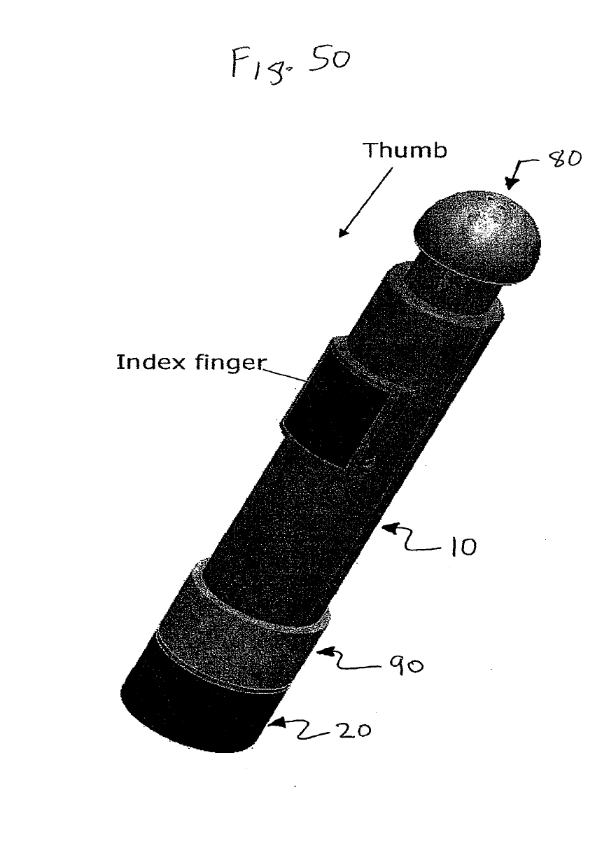

With reference to FIGS. 47-48, it can be seen that the front cap 20 includes a skin contacting surface 20a which includes a lancet needle opening 20b sized and located to allow one of the lancet needles LN to pass or extend therethrough. The front cap 20 has an outer generally cylindrical surface 20c, an annular rear end 20b, and a generally cylindrical inner surface 20e which is sized and configured to slidably engage with outer surface 90B5 of the inner sleeve 90B. Although not shown, the front cap 20 and inner sleeve 90B preferably include mechanisms, i.e., a recess and projection, to ensure that the front cap 20 is position in only one rotational position relative to the inner sleeve 90B. This ensures that the lancet opening 20b is properly position relative to the pushing member 40b which, in turn, ensures that the lancet needle LN of the lancet L being moved to the extended position (see FIG. 4) passes through the opening 20b.

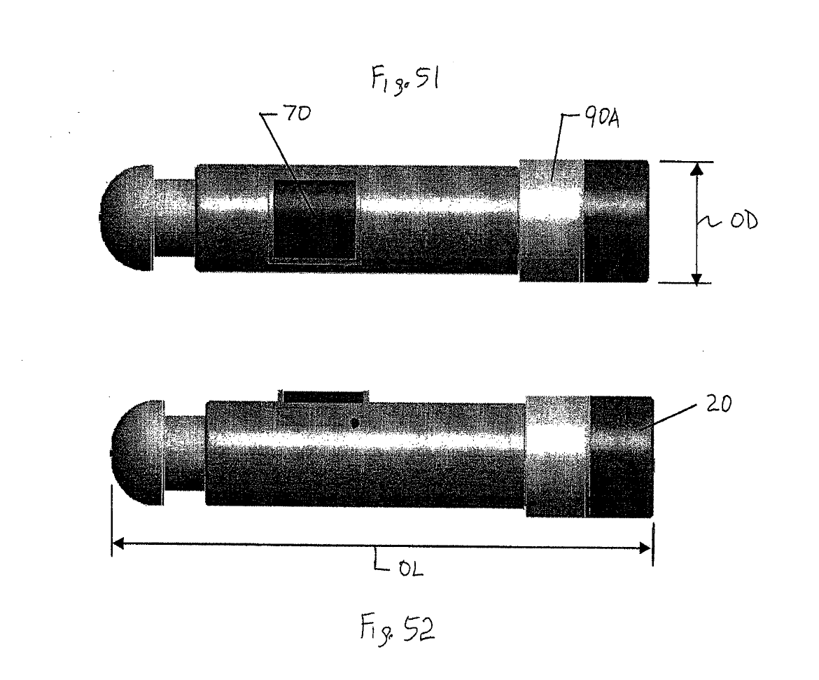

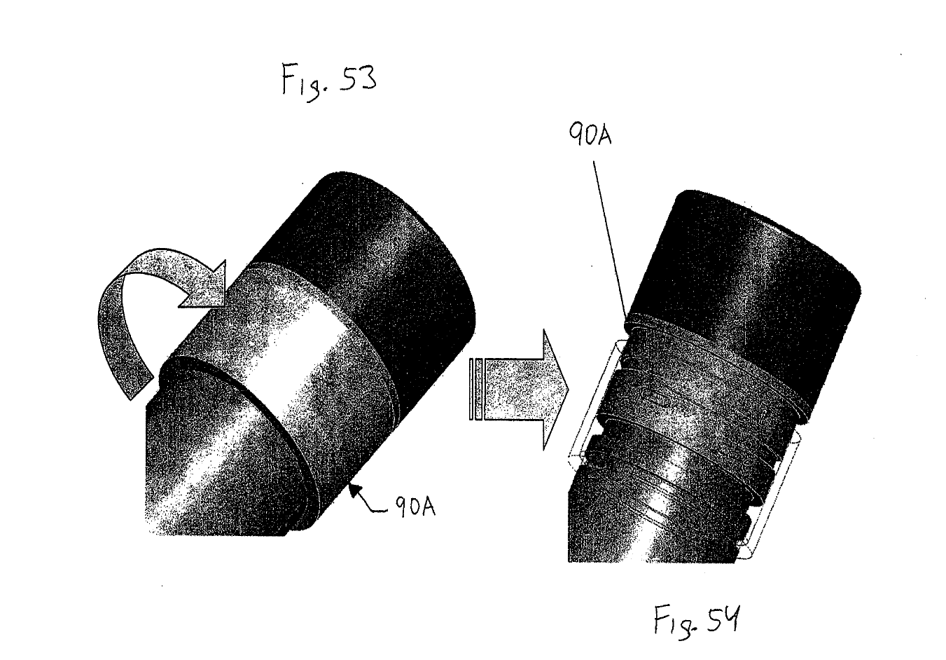

FIGS. 50-54 shows various views of the lancet device LD shown in FIG. 1 and illustrates how the lancet device can be armed, triggered, and have depth of penetration adjusted. As FIG. 50 demonstrates, the user can place his or her thumb on the rear button portion of the arming member 80 and depress it in the direction of arrow. Then, the user can depress the trigger 70 with the user's index finger to cause the lancet device LD to automatically move one of the lancets L to the extended or puncturing position (see FIG. 4), thereby arming and triggering the lancet LD with only one hand. When the user desires to adjust a depth of penetration of the lancet device LD, the user grip and cause rotation of the depth adjustment system 90 (see FIGS. 53 and 54) to thereby cause axial movement of the front cap 20 relative to the housing 10. FIGS. 51 and 52 show two side views of the lancet device LD shown in FIG. 50 with FIG. 52 showing the lancet device LD of FIG. 51 rotated upwards by about 90 degrees. By way of non-limiting example, the lancet device front end portion can have an outer diameter OD of about 21 mm, and the lancet device LD can have an over length OL of about 120 mm.

FIG. 55 shows a side cross-section view of the cartridge assembly 30 which can be used in the lancet device LD shown in FIG. 1, and illustrate how an axis LBA of the main body portion of each lancet L is axially offset from a center axis LNA of the lancet needle LN and that both of these axes are axially offset from a center axis CA of the cartridge assembly 30.

Although not shown, one or more of the parts of the lancet device LD such as, e.g., the housing 10, can preferably made transparent and/or translucent so that a user will clearly be able to see internal components.

The invention contemplates embodiments which provide for the removal of the front assembly 90 and front cap 20 as a unit from the housing 10 in order to allow for insertion and removal of the cartridge assembly 30, or alternatively for the removal of only the front cap 20 from the depth adjustment system 90 in order to allow for insertion and removal of the cartridge assembly 30 from the dousing 10. Furthermore, the invention can be used in the following ways; the user can load and trigger the lancet device LD more than once without advancing the cartridge assembly 30 (via arming member 80) to the next rotational position, or the user can use a lancet L only once and then rotate the cartridge assembly 30 to the next position. For safety reasons, the former option should only be used by the same user and when the lancet L being reused was last used only mere hours ago. Finally, the invention also contemplates using the arming member 80 to cause an ejection of the cartridge assembly 30 when the arming member 80 is move into the housing 10 and when the cartridge assembly is rotated to a final rotational position.

It is also possible to utilize an indicator system to inform the user which lancet needle has already been used and/or how many new or unused lancets remain in the cartridge assembly 30. Non-limiting examples of such system can be found in US 2007/0233167 to WEISS et al., the entire disclosure of which is hereby expressly incorporated by reference in its entirety. All the parts of the lancet device LD, with the exception of the springs and needles (which can respectively be made of spring steel and stainless steel), may be made from plastic materials and can be formed using conventional injection molding techniques or other known manufacturing methods. Bay way of non-limiting example, all or most of the parts such as the housing, trigger, trigger slide, arming member, cartridge body, can be made of ABS plastic with the exception of the springs (which can be stainless steel) and the plunger assembly which can be made of polyoxymethylene (Delrin plastic). However, when practical, other materials and manufacturing processes may also be utilized.

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed is:

1. A cartridge for a device, the cartridge comprising:
a generally cylindrical cartridge body;
a plurality of generally axially movably mounted lancets or lancet needles; and
a plurality of biasing members, each adapted to apply a biasing force to one of the lancets or lancet needles.

2. The cartridge of claim 1, further comprising a mechanism allowing the cartridge to be mounted to the device.

3. The cartridge of claim 1, wherein the device comprise at least one of a lancet device, a testing device, and a glucose meter.

4. The cartridge of claim 1, wherein the cartridge is one of adapted to rotate about a central axis and rotate in only one direction about a central axis.

5. The cartridge of claim 1, wherein at least one of:
each lancet or lancet needle is individually moved to an extended position by a pushing device which merely contacts and pushes the lancet or lancet needle without becoming connected to the lancet or lancet needle in any way, temporarily or otherwise; and
needles of the of the lancets or lancet needles are axially oriented in a manner which is offset from a main axis of each lancet or lancet needle.

6. The cartridge of claim 1, wherein the biasing members comprise springs, each spring having one end adapted to contact one of the plurality of lancets or lancet needles.

7. The cartridge of claim 1, wherein the cartridge comprises an axial length which is greater than an outer diameter.

8. The cartridge of claim 1, wherein the cartridge comprises an outer diameter of no greater than about 1 inch.

9. The cartridge of claim 1, wherein the cartridge comprises an axial length of no greater than about 1.5 inches.

10. The cartridge of claim 1, wherein each lancet or lancet needle comprises an opening or recess which receives therein a portion of one of the biasing members.

11. The cartridge of claim 1, wherein each of the plurality of lancets or lancet needles comprises a synthetic resin head portion and a metal needle portion.

12. The cartridge of claim 1, wherein the cartridge comprises a plurality of equally angularly spaced openings each sized to receive therein one of the lancets or lancet needles.

13. The cartridge of claim 1, further comprising a centrally arranged opening sized to receive therein a device for causing rotation of the cartridge.

14. The cartridge of claim 1, further comprising a centrally arranged opening comprising at least one of an axial groove and an axial projection adapted to engage with a device for causing rotation of the cartridge.

15. The cartridge of claim 1, further comprising a centrally arranged opening comprising axial grooves and projections adapted to engage with a spline of a device for causing rotation of the cartridge.

16. The cartridge of claim 1, further comprising a plurality of openings arranged on back end of the cartridge, each opening being sized to receive therein a device for causing one of the lancets or lancet needles to move to an extended or puncturing position.

17. The cartridge of claim 16, wherein the device for causing one of the lancets or lancet needles to move to an extended or puncturing position contacts a rear-most end of the one of the lancets or lancet needles.

18. The cartridge of claim 16, wherein the one of the lancets or lancet needles automatically moves back to a retracted position when the device for causing one of the lancets or lancet needles to move to an extended or puncturing position is moved to a retracted position.

19. The cartridge of claim 1, further comprising an alignment mechanism allowing the cartridge to be mounted to the device in only a single position.

20. The cartridge of claim 1, further comprising one of a notch, a groove, and a projection which ensures that the cartridge is mounted to the device in a predetermined position.

21. The cartridge of claim 1, further comprising a locking mechanism preventing rotation of the cartridge once the cartridge has rotated in the device to a locking position.

22. The cartridge of claim 1, further comprising a projection which prevents rotation of the cartridge after the cartridge has rotated in the device to a final position.

23. A method of puncturing a surface of skin using a device comprising the cartridge of claim 1, the method comprising:
arranging the device adjacent against a user's skin;
triggering the device so that one of the plurality of lancets or lancet needles is caused to penetrate the user's skin; and
rotating the cartridge to another position.

24. A cartridge for a device, the cartridge comprising:
a generally cylindrical-shaped body;
a plurality of axially oriented lancet needles;
a plurality of springs; and
each spring being structured and arranged to move one of the plurality of lancet needles towards a retracted position,
wherein the cartridge is structured and arranged to be at least one of rotatably mounted, removably mounted, and non-axially movably mounted to the device.

25. The cartridge of claim 24, wherein the generally cylindrical-shaped body comprises a center opening and a plurality of axially oriented and equally angularly spaced openings, wherein the plurality of axially oriented lancet needles are movable along a generally linear path without substantially rotating, and wherein the center opening is adapted to receive a device for causing rotation of the cartridge.

26. A method of puncturing a surface of skin using a device comprising the cartridge of claim 24, the method comprising:
arranging the device adjacent against a user's skin;
triggering the device so that one of the plurality of lancet needles is caused to penetrate the user's skin; and
rotating the cartridge to another position.

27. A lancet device comprising:
a housing comprising a trigger; and
a generally cylindrically-shaped cartridge comprising a plurality of lancet needles and a plurality of springs and being adapted to rotate between different positions.

28. The lancet device of claim 27, wherein the cartridge comprises a center opening which is sized to receive therein a portion of a device for causing rotation of the cartridge.

29. The lancet device of claim 27, wherein the plurality of lancet needles are independently movably mounted.

30. The lancet device of claim 29, wherein the plurality of lancet needles are generally axially oriented.

31. The lancet device of claim 27, wherein the cartridge is removably mounted to the housing.

32. The lancet device of claim 27, wherein the cartridge is insertable into the housing in only one rotatable position and removable from the housing in only one rotatable position.

33. The lancet device of claim 27, wherein the cartridge is removable from the housing in only a final rotatable position after all of the lancet needles have been utilized.

34. A device comprising:
a housing;
a cartridge comprising a plurality of lancet needles;
the cartridge being rotatably mounted to a front end of the housing;
a mechanism which prevents axial movement of the cartridge after the cartridge is mounted to the front end of the housing; and
a device rotatably mounted to the housing which allows a user to rotate the cartridge between a plurality of different positions,
wherein the device has one end which extends outside of a rear end of the housing and is adapted to be gripped by the user to cause rotation thereof.

35. The device of claim 34, wherein the housing comprises a generally cylindrically shaped two-piece housing.

36. The device of claim 34, further comprising a mechanism which retains the cartridge in at least one rotational position.

37. The device of claim 34, wherein the device which allows a user to rotate the cartridge between a plurality of different positions comprises a spline and is axially movable at least between an initial position and a position which places the device in an armed or trigger-set position.

38. The device of claim 34, further comprising a mechanism which retains the cartridge in at least one rotational position, wherein the mechanism which retains the cartridge in at least one rotational position comprises a deflecting member arranged on the device rotatably mounted to the housing.

39. The device of claim 34, wherein the device which allows a user to rotate the cartridge between a plurality of positions is biased towards an initial position.

40. The device of claim 34, further comprising an alignment mechanism allowing the cartridge to be initially mounted within the front end of the housing in only a single position.

41. The device of claim 34, further comprising a deflectable projection which ensures that the cartridge is mounted to the housing in a predetermined position.

42. The device of claim 34, further comprising guiding grooves arranged in the front of the housing for allowing insertion and rotation of the cartridge.

43. The device of claim 34, wherein the cartridge and housing comprise an arrangement which prevents rotation of the cartridge after the cartridge has rotated in the housing to a predetermined position.

44. The device of claim 34, further comprising a depth adjustment cap arranged at the front end of the housing.

45. A method of puncturing a surface of skin using the device of claim 34, the method comprising:
arranging the device adjacent against a user's skin;
triggering the device so that one of the plurality of lancet needles is caused to penetrate the user's skin; and
rotating the cartridge to another position.

46. A method of assembling a device for puncturing a skin surface, the method comprising:
installing a cartridge comprising a plurality of lancets and lancet springs at least partially within a device housing,
whereby the cartridge, once installed, is capable of being rotated between at least two different positions.

47. A method of using a device for causing a puncture in a skin surface, the method comprising:
installing a cartridge comprising a plurality of lancets and lancet springs at least partially within a device housing;
triggering the device so that one of the plurality of lancet needles is caused to penetrate the user's skin; and
rotating the cartridge to another position.
